(19) United States
(12) Patent Application Publication
(10) Pub. No.: US 2026/0112495 A1
(43) Pub. Date: Apr. 23, 2026

(54) FULLY AUTONOMOUS MEDICAL SOLUTION

(71) Applicants: Mazen A. Al-Sinan, Dhahran (SA); Ho-Hsun David Kuo, Flagstaff, AZ (US)

(72) Inventors: Mazen A. Al-Sinan, Dhahran (SA); Ho-Hsun David Kuo, Flagstaff, AZ (US)

(21) Appl. No.: 19/423,922

(22) Filed: Dec. 17, 2025

Related U.S. Application Data
(63) Continuation-in-part of application No. 17/483,558, filed on Sep. 23, 2021.
Application No. 17/483,558 is a continuation-in-part of application No. 16/904,219, filed on Jun. 17, 2020, now abandoned.
Application No. 17/483,558 is a continuation-in-part of application No. PCT/US21/23661, filed on Mar. 23, 2021.
Application No. PCT/US21/23661 is a continuation-in-part of application No. 16/904,219, filed on Jun. 17, 2020, now abandoned.
(60) Provisional application No. 62/993,437, filed on Mar. 23, 2020.
(60) Provisional application No. 62/993,437, filed on Mar. 23, 2020.

(51) Int. Cl. G16H 50/20; G16H 10/40; G16H 10/60; G16H 20/10; G16H 40/63; G16H 70/60; G16H 80/00

(52) U.S. Cl. CPC G16H 50/20; G16H 10/40; G16H 10/60; G16H 20/10; G16H 40/63; G16H 70/60; G16H 80/00

(57) ABSTRACT

The integration of various services for diagnosis and treatment determination of a disease in a patient into a platform within a computing device. The platform may provide the User with a diagnosis of abnormalities through dialogue and provide prescriptions and treatment plans. The platform includes an external communicator module for communicating with a plurality of external medical sources to compile the user's medical records into a single format. The platform further includes a diagnosis module that uses the user's medical records and a database of all diseases and symptoms to automatically diagnose an ailment in a user and treatment options for said ailment. The platform further includes a case monitoring module for tracking the recovery time of the user and adjusting the recovery time if a change in treatment is executed.

### CROSS-REFERENCES TO RELATED APPLICATIONS

This application is a continuation-in-part and claims benefit of U.S. patent application Ser. No. 17/483,558, filed Sep. 23, 2021, which is a continuation-in-part and claims benefit of U.S. Non-Provisional patent application Ser. No. 16/904,219, filed Jun. 17, 2020, which claims benefit of U.S. Provisional Patent Application No. 62/993,437, filed Mar. 23, 2020, the specifications of which are incorporated herein in their entirety by reference.

U.S. patent application Ser. No. 17/483,558 is also a continuation-in-part and claims benefit of PCT Application No. PCT/US21/23661 filed Mar. 23, 2021, which is a continuation-in-part and claims benefit of U.S. patent application Ser. No. 16/904,219, filed Jun. 17, 2020, and claims benefit of U.S. Provisional Application No. 62/993,437 filed Mar. 23, 2020, the specifications of which are incorporated herein in their entirety by reference.

### FIELD OF THE INVENTION

The present invention is directed to the diagnosis and treatment determination of a disease in a patient within a computing device.

### BACKGROUND OF THE INVENTION

The present invention is in the technical field of intelligent medical automation. More particularly, the present invention is in the technical field of autonomous medical solutions to diagnose and provide treatment to an individual. There are many challenges with the current practices for seeking medical care. When an individual experiences an abnormality in their health condition, one of the following scenarios could take place:
1. Seeing a physician while the condition might be minor and temporary and doesn't require seeing a physician or the problem could be cured by taking over-the-counter drugs or by non-drug treatment. However, identifying the suitable over-the-counter drug is challenging.
2. Ignoring the symptoms or taking over-the-counter drugs while there is a genuine need to see a physician, considering that many people avoid seeing physicians for various reasons; such as financial expenses, time constraints, or fear of seeing a physician.

As a result, seeing a physician while there is no need is a waste of resources, while avoiding seeing one when it is necessary could aggravate the condition and endanger the life of the patient.

Furthermore, many individuals live alone and in case of a need for medical care, especially when they are sick, might find ordering and collecting an over-the-counter drug, identifying appropriate medical centers, setting up an appointment with a physician, or, in severe cases, calling for an ambulance could be challenging.

Another issue is that medical illness could occur at any time while physicians are only available during working hours unless the patient decides to go to the emergency room. Going to the emergency in many cases could be avoided; the patient may simply need temporary relief or someone to advise him/her whether there is an urgent need to go to the emergency or not.

For example, elderly people and those who might be susceptible to heart attacks or a crucial need for emergency medical intervention should be monitored around the clock by someone such as a home nurse or an accompanying party who should call for an ambulance in case of any abnormalities in vital signs that necessitate urgent medical intervention.

Furthermore, physicians usually encounter challenges in diagnosing diseases since many of them have similar or nonspecific signs and symptoms. For instance, red spots on the skin could be a sign of a kidney problem. A physician could diagnose a condition based on experience or results of many tests and examinations which might be unnecessary. Physicians depend on their experience and the results of tests and examinations to diagnose any medical condition. However, Artificial Intelligence (A.I.) and Machine Learning (M.L.) could outperform physicians in diagnosing a patient's condition provided the results of tests and examinations are fed.

On the other hand, a patient's medical historical record is important for the physician to diagnose and prescribe the appropriate treatment. Reviewing the medical history is time-consuming to the physician and much important information relevant to the patient's condition could be overlooked. In case of an emergency, the physician would take action without reviewing the medical records of the patient. Similarly, when the medical records are unavailable, they might request unnecessary tests or proceed with prescribing a treatment regardless of the patient's medical historical record. A physician could also prescribe a medicine hoping that it will resolve the problem and in the follow-up appointment which is usually after one week the physician could discover that the medicine is not working and has to find an alternative medication or treatment.

In some cases, a patient might need temporary home nursing care such as in case of severe flu where the patient's condition does not require admission to the hospital but still requires someone to help them, such as a certified nurse if they live alone. Finding a freelance nurse within a short duration in these cases could be difficult.

All the above challenges have substantial impacts from wasting resources by taking unnecessary tests to see a physician while the problem could be resolved with an over-the-counter drug to exposing the patient to a fatal risk or aggravation of the patient health condition because of the delay in seeking the medical care while there is an imminent need for medical intervention.

In a situation similar to the Covid-19 pandemic, the proposed invention could sustain medical services for most routine medical care services, especially in the case of conditions that do not require performing medical procedures. The stretched medical resources could focus on the pandemic while the patient could still receive medical services while avoiding going to the medical centers that could be risky because of the pandemic. It could also provide thousands of self-isolated patients with necessary medical consultations on the spot.

A.I. has been previously utilized in diagnosis and health services, but the fundamental challenges in medical services have not been surmounted proportionally to the advancement in A.I. applications in general and in health services in particular. There is a potential to enhance the quality and optimization of resources in medical services throughout by applying A.I. technology in an integrated manner. Technologies such as Blockchain, Machine Learning (M.L.), Internet of Things (IoT), Natural Language Processing (NLP), and Data Science could additionally be integrated and employed to have one unified synchronized platform that provides a full medical solution to individuals.

The present invention utilizes artificial intelligence, machine learning, medical history, and natural language for interaction with the system to provide medical diagnosis and treatment determination without the presence of a physician.

Prior systems teach virtual physician platforms capable of accepting user symptoms and generating an accurate diagnosis and treatment for the disease-causing the said symptoms. However, none of the prior systems teach an autonomous system capable of acting without any intervention by medical professionals while still generating solutions on par or exceeding the accuracy and time-efficiency of a medical professional. For example, U.S. Publication No. 2019/0244683 (hereinafter “Francois”) provides a computer program product for assembling a database comprising electronic medical records (EMRs). These EMRs must be filled in by a third-party medical professional with information that the medical professionals fill in manually. Additionally, Francois is only able to process information available from public records, while the present invention will have access to all medical records, including those privately stored at hospitals, pharmacies, etc. Furthermore, Francois is unable to efficiently encrypt their data due to a lack of data compression, requires connection to a separate application for dietician and wellbeing module capabilities, utilizes self-administered monitoring tests, and requires medical professional intervention to request a prescription in accordance with a treatment. The present invention efficiently compresses data, incorporates the dietician and wellbeing modules into the platform, utilizes autonomously monitored and collected test results into the back office for evaluation and to provide treatment, and is able to request the prescription on behalf of the user for a total hands off treatment. In summary, the difference between the prior arts and the proposed invention is that the exchange of data in the prior arts is triggered by human beings while in the proposed solution the exchange of data occurs autonomously.

### BRIEF SUMMARY OF THE INVENTION

It is an objective of the present invention to provide systems that allow for the integration of various services under one platform and the diagnosis and treatment determination of a disease in a patient within the platform, as specified in the independent claims. Embodiments of the invention are given in the dependent claims. Embodiments of the present invention can be freely combined with each other if they are not mutually exclusive.

The present invention features a software platform. The platform may comprise an external communicator module. The external communicator module may comprise a data encryption module for accepting data from a plurality of sources and encrypting the data into a standard format. The data encryption module may accept information from a user, or a plurality of external medical sources. The external communicator module may further comprise a data compression module for compressing and transmitting encrypted data. The data compression module may transmit data to the plurality of external medical sources or an external storage component. The plurality of external medical sources may comprise a hospital, a clinic, a lab, an emergency center, and a pharmacy.

The software platform may additionally feature a diagnosis module. The diagnostic module may comprise a database of disease symptoms mapped to possible causes. The diagnostic module may comprise instructions for accessing the database of disease symptoms and accepting a list of user symptoms from the user. The diagnostic module may further comprise generating a table to map each user symptom to all possible causes for each symptom and generating a second table displaying all possible causes that share every user symptom. The diagnostic module may further comprise accepting data from a database of user medical records and generating a probability table ranking all possible causes based on the probability of the presence of each possible cause. Finally, the diagnostic module may determine a treatment for the possible causes based on information from the database of medical records. In some embodiments, the diagnostic module may additionally request tests from the user to better determine the possible cause.

The software platform of the present invention may additionally feature a case monitoring module. The case monitoring module may comprise instructions for monitoring the user's health and recovery as the user carries out a treatment determined by the diagnostic module. The case monitoring module may further comprise instructions for reminding the user to carry out a prescription related to the treatment, comparing an expected recovery time to an actual recovery time to determine if a secondary course of action is necessary, adjusting the prescription as the secondary course of action, and adjusting the expected recovery time based on the secondary course of action.

Note that natural language is used in the front end of the software for the interface with the user, and AI and data mining is used in the back end. Users will describe their symptoms to the software with no medical professional involved. The back end analysis is based on an Artificial Intelligent system with self learning to improve searching the medical database to determine the best method to provide treatment to the user. Data transmitted is from the front end interface and the back office (external data sources). All information is encrypted due to medical information requirements and compressed due to the home user's internet bandwidth. The software may have its own encryption methodology.

One of the unique and inventive technical features of the present invention is the encryption of data from both the user and a plurality of external medical sources into a standard format that can be used interchangeably by both parties. Without wishing to limit the invention to any theory or mechanism, it is believed that the technical feature of the present invention advantageously provides for a comprehensive source of a user's medical history and conditions to allow for more efficient diagnosis of user issues, and allows the software platform to send diagnosis information to the external medical sources for later archiving and use. The autonomous aspect of the feature provides for a time and resource-efficient manner of compiling and transmitting the user's medical history. None of the presently known prior references or work has the unique inventive technical feature of the present invention.

Furthermore, the feature of the present invention is counterintuitive. The reason that it is counterintuitive is because the feature contributed to a surprising result. One skilled in the art would expect the conversion of data to and from a plurality of different formats between the user and the plurality of external medical sources to require a large amount of computation and time to execute properly, making it more efficient overall to simply view, compile, and transmit the data manually. Surprisingly, the present invention is capable of viewing, compiling, and transmitting data between a plurality of sources and a plurality of formats efficiently by generating a script for each encountered format to quickly execute for future instances of the same formats. Thus, the feature of the present invention contributed to a surprising result and is counterintuitive.

Another unique and inventive technical feature of the present invention is the use of a database of disease symptoms and a user's medical history to automatically diagnose a medical issue and generate treatment options for the diagnosis. Without wishing to limit the invention to any theory or mechanism, it is believed that the technical feature of the present invention advantageously provides for an efficient, comprehensive, and accurate form of automatic diagnosis and treatment that the user can execute from the comfort of home. None of the presently known prior references or work has the unique inventive technical feature of the present invention.

Furthermore, the feature of the present invention is counterintuitive. The reason that it is counterintuitive is because it contributed to a surprising result. For example, one skilled in the art would expect that the system would be unable to handle a situation in which a user reports symptoms that correspond to more than one disease, even given the information from the disease database and the user's medical records. Surprisingly, this situation is overcome by the MyDoc platform through the use of a probability table that lists the most likely matches for the user's condition through the use of the user's medical history and additional tests requested by the diagnostic module, providing for an accurate diagnosis a mass majority of the time. Thus, the feature of the present invention contributed to a surprising result and is counterintuitive.

Another unique and inventive technical feature of the present invention is the prediction of recovery time for a treatment and the adjustment of the recovery time upon application of a secondary course of action. Without wishing to limit the invention to any theory or mechanism, it is believed that the technical feature of the present invention advantageously provides for an efficient and accurate way for the user to plan out their recovery time and keep track of whether or not their treatment is progressing at the correct pace. None of the presently known prior references or work has the unique inventive technical feature of the present invention.

Furthermore, the feature of the present invention is counterintuitive. The reason that it is counterintuitive is because it contributed to a surprising result. For example, one skilled in the art would expect the planned recovery time to be entirely inaccurate in certain cases, such as when a medication that a user is already taking quickens or slows recovery from an average recovery time in a database. Surprisingly, the present invention can predict recovery times for a specific user accurately through the use of active monitoring and calculations that personalize recovery times to the user, resulting in a more accurate prediction. Thus, the feature of the present invention contributed to a surprising result and is counterintuitive.

Another one of the unique and inventive technical features of the present invention is the implementation of a fully autonomous system for disease diagnosis and treatment. Without wishing to limit the invention to any theory or mechanism, it is believed that the technical feature of the present invention advantageously allows for an autonomous, AI-driven virtual physician designed to diagnose, treat, and monitor users in their homes without human intervention. The present invention's independence from medical professionals is evident in its unique modules, which perform a range of tasks, from gathering medical history to providing prescriptions and emergency response. The autonomous nature of the present invention is supported by Diagnostic and Treatment Modules for diagnosing medical conditions and autonomously creating treatment plans, Monitoring and Adaptive Treatment processes comprising continuously monitoring patient recovery and autonomously adjusting treatment plans, and Emergency Response capabilities, where emergencies are detected and emergency services are automatically contacted. None of the presently known prior references or work has the unique inventive technical feature of the present invention.

Another one of the unique and inventive technical features of the present invention is the implementation of autonomous communication with external sources for rapid aid of patients. Without wishing to limit the invention to any theory or mechanism, it is believed that the technical feature of the present invention advantageously allows for a patient to receive aid in urgent situations without the need to dial a number manually. This is useful for situations in which a patient is alone and unable to access a phone to dial for help due to physical and/or mental hindrances. This is also useful for situations in which all nearby parties are occupied, no phones are available, or any other scenario that would interfere with contacting emergency services. Aligning with the vision of the present invention for a fully autonomous virtual physician software, this feature allows for fully autonomous identification of urgent patient events and contact of emergency services to aid said patient as quickly as possible. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a schematic of an external communicator module of the present invention.

FIG. 2 shows a schematic of a diagnosis module of the present invention using the medical records and user symptoms to generate a list of possible causes for the user symptoms.

FIG. 3 shows a schematic of the diagnosis module of the present invention using a database of disease information to create a plurality of tables and generate a list of possible causes for the user symptoms.

FIG. 4 shows a schematic of the software platform of the present invention.

FIG. 5 shows a block diagram of the subject matter illustrating a general scheme of the Fully Autonomous Medical Solution for Individuals.

FIG. 6 shows a block diagram of the User Account Module and its integration with the whole invention.

FIG. 7 shows a block diagram of the General Information.

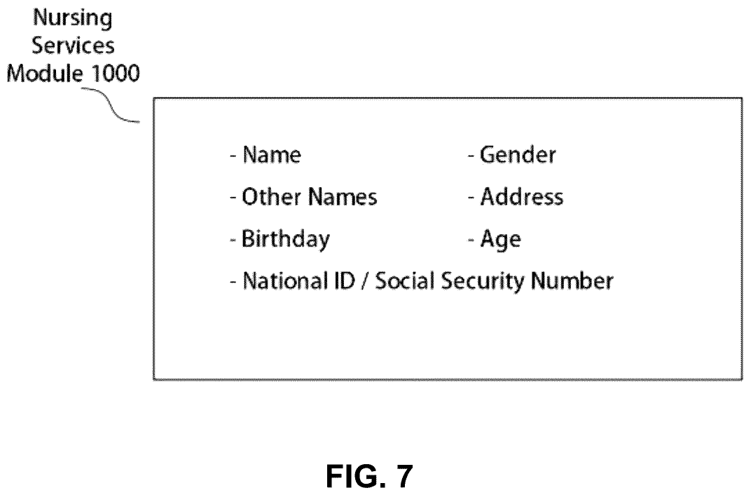

FIG. 8 shows a block diagram of the Medical Records.

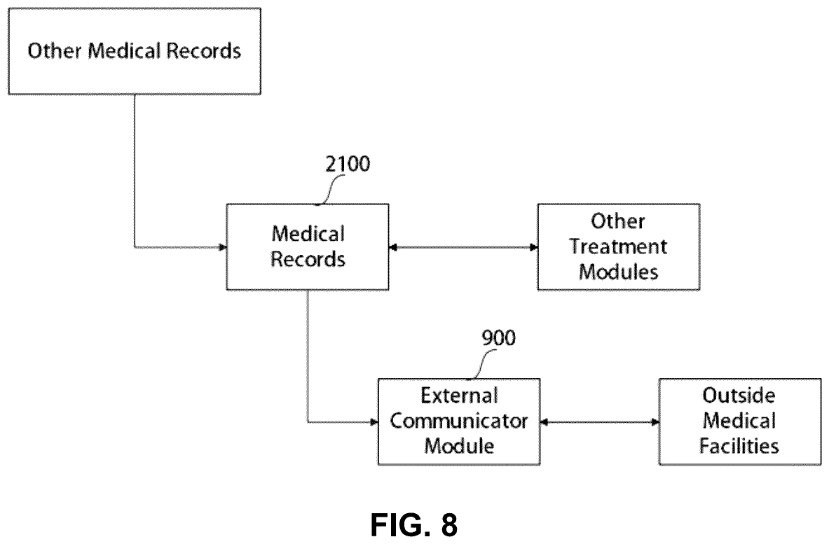

FIG. 9 shows a block diagram of the Reported conditions.

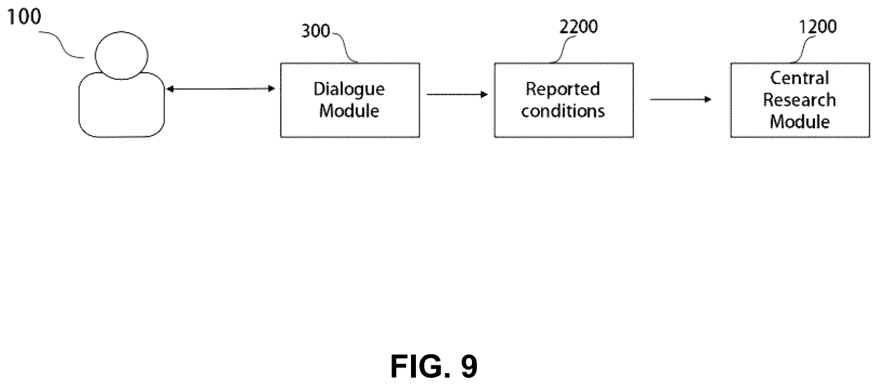

FIG. 10 shows a block diagram of the User Habits.

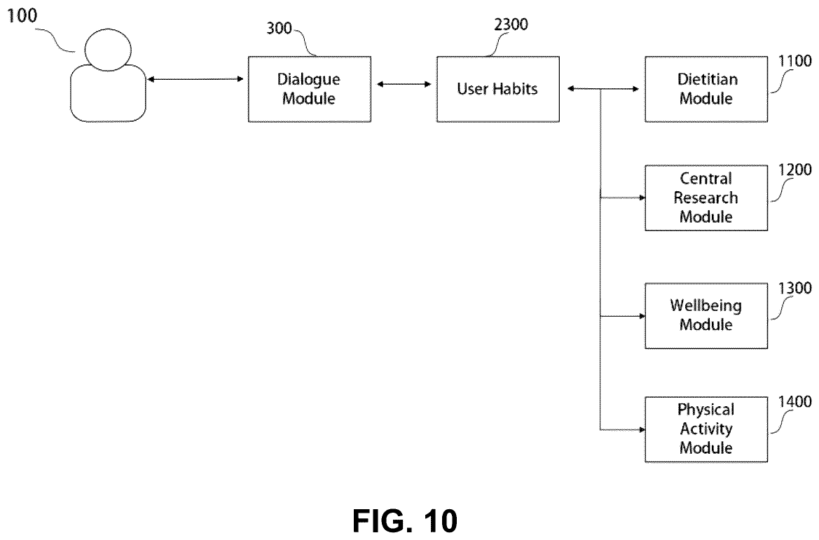

FIG. 11 shows a block diagram of the Payment Gateway.

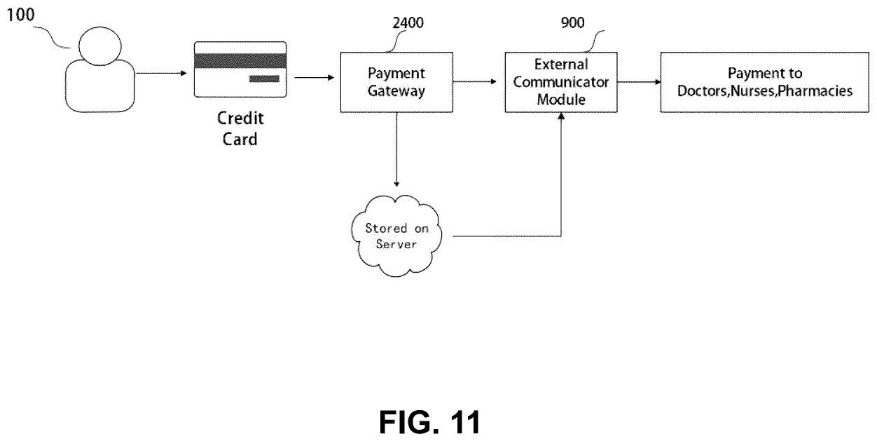

FIG. 12 shows a block diagram of the Dialogue Module.

FIG. 13 shows a block diagram of the sample greetings of the Dialogue Module.

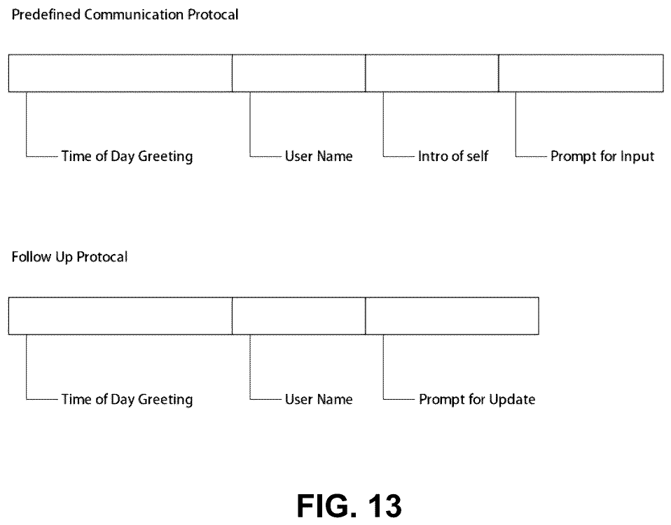

FIG. 14 shows a block diagram of the Diagnostic Module.

FIG. 15 shows a block diagram of the property analysis of the Diagnostic Module.

FIG. 16 shows a block diagram of the Test Analyzer Module.

FIG. 17 shows a block diagram of the Treatment Module.

FIG. 18 shows a block diagram of the Case Monitoring Module.

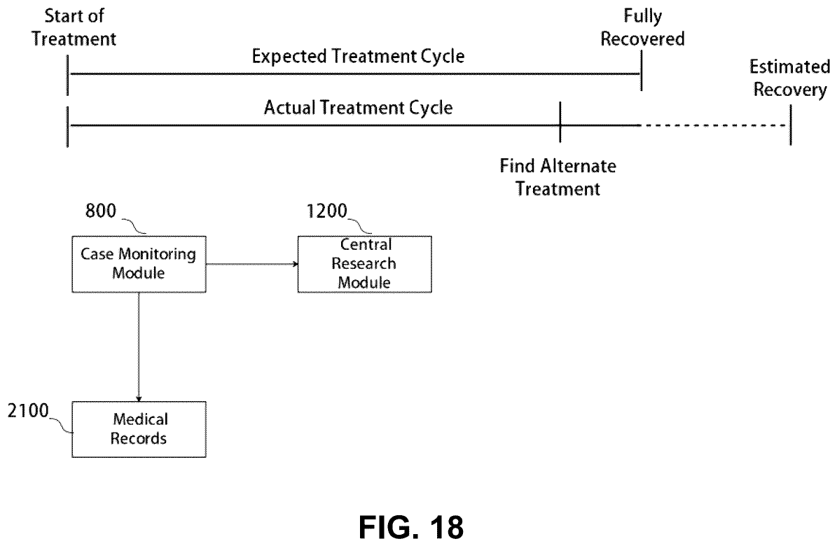

FIG. 19 shows a block diagram of the External Communicator Module.

FIG. 20 shows a block diagram of the Nursing Services Module.

FIG. 21 shows a block diagram of the Dietitian Module.

FIG. 22 shows a block diagram of the Central Research Module.

FIG. 23 shows a block diagram of the Wellbeing Module.

FIG. 24 shows a block diagram of the Physical Activity Module.

FIG. 25 shows a block diagram of the Breathing Apparatus Module.

FIG. 26 shows a block diagram of the Defibrillator Module.

FIG. 27 shows a block diagram of the Psychological Therapy Module.

### DETAILED DESCRIPTION OF THE INVENTION

Following is a list of elements corresponding to a particular element referred to herein:
100 user
200 user account module
300 dialogue module
400 diagnostic module
410 first table
420 second table
430 probability table
450 list of possible causes
475 treatment options
500 test analyzer module
600 software platform
700 treatment module
800 case monitoring module
900 external communicator module
910 data encryption module
920 data compression module
930 plurality of external medical sources
1000 nursing services module
1100 dietician module
1200 central research module
1300 wellbeing module
1400 physical activity module
1500 breathing apparatus module
1600 defibrillator module
1700 psychological therapy module
2000 general information
2100 medical records
2200 reported condition
2300 user habits
2400 payment gateway
9000 external storage component

As stated throughout the present application, the term “autonomous” refers to noninvolvement and nonintervention by medical or support personnel in the operation of the software platform. Fully autonomous in the front end interface in communicating with the user utilizing Natural Language to gather information/additional information, perform tests and obtain enough information for the back end Artificial Intelligence to perform the necessary analysis to obtain the treatment plan. The treatment is monitored autonomously and treatment progress is evaluated in comparison with average recovery results. If the recovery is not as expected or slow in comparison. The software will initiate a new treatment plan for the user and repeat the monitoring process to ensure the user has a good recovery.

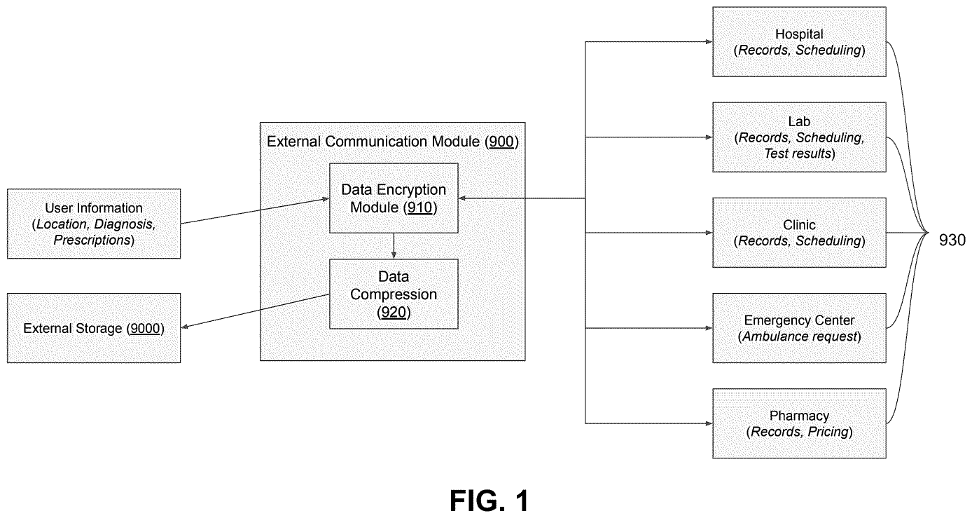

Referring now to FIG. 1, the present invention features a virtual physician platform (600) for compiling a user's medical history from a plurality of sources. In some embodiments, the platform may comprise an external communicator module (930) for gathering information pertaining to the user's medical history from a plurality of sources. The external communicator module (900) may comprise a data encryption module (910) with instructions for accepting data from a source, and encrypting the data into a standard format. The data encryption module (910) is capable of accepting data directly from the user, or a plurality of external medical sources (930). In some embodiments, the process is for a person to process the data for each hospital/format/medical group, and the platform may learn from each format and process the conversion procedure as a script and repeat the process for all future information of the same format. For example, if data format A needs to be converted to data format Z, a person or the system utilizing machine learning may process the data to pull out the personal information, medical records, etc. The location of the information and how it is stored as data format A may be stored and all future data format A can be converted the same way. This process may be repeated for all other formats. For image data, the images may be exported from the source and processed by the platform by using machine learning to read and analyze the images. Images may be formatted into Nifti or Diacom.

The external communicator module (900) may further comprise a data compression module (920) having instructions for accepting encrypted data from the data encryption module (910), compressing the encrypted data into compressed data, and transmitting the compressed data to an external storage (9000) component or the plurality of external medical sources (930). The plurality of external sources (930) may be a hospital, a medical lab, a clinic, an emergency center, and a pharmacy. The external communicator module (900) may communicate with the hospital to receive medical records of the user and scheduling information for arranging an appointment and transmits the user data and appointment requests to the hospital. The external communicator module (900) may communicate with the medical lab to receive medical records of the user, scheduling information for arranging an appointment, and lab results, and transmits the user data and appointment requests to the medical lab. The external communicator module (900) may communicate with the clinic to receive medical records of the user and scheduling information for arranging an appointment, and transmits the user data and appointment requests to the clinic. The external communicator module (900) may communicate with the emergency center to request an ambulance, and transmits the user data and a location of the user to the emergency center. The external communicator module (900) may communicate with the pharmacy to receive medical records of the user and pricing information for prescriptions and transmits the user data, a plurality of prescriptions, and a delivery location for prescriptions to the pharmacy. The pricing information may comprise a pricing chart of various pharmacy locations, prices, ratings, reviews, brands, etc. Additionally, the treatment module (700) may provide a list of one or more pharmacies chosen by the user and/or determined by the software platform (600) through location detection/proximity.

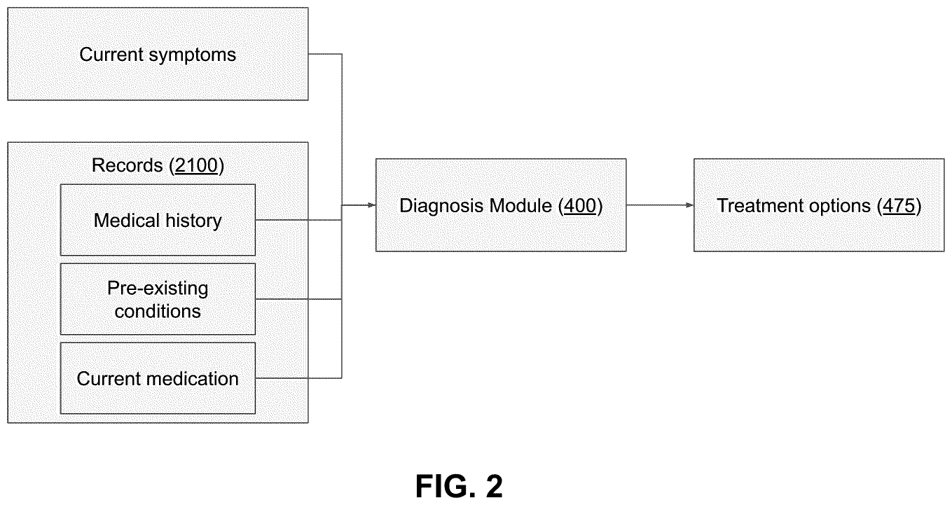

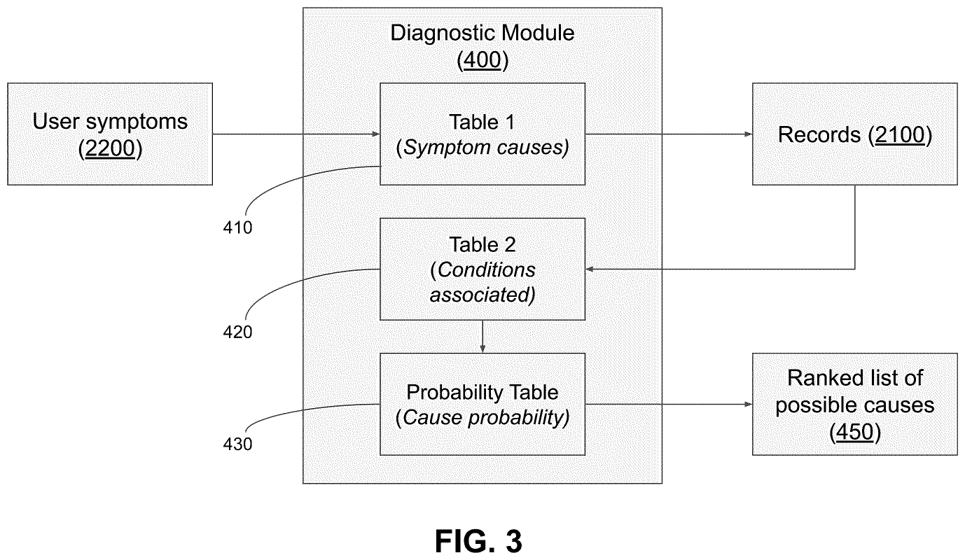

Referring now to FIGS. 2-3, the present invention features a software platform for diagnosing a medical issue of a user. In some embodiments, the platform may comprise a database of medical records (2100). The database of medical records (2100) may comprise a medical history of the user, a list of pre-existing conditions of the user, and a list of medications currently taken by the user. The platform may further comprise a diagnostic module (400) having instructions for accessing a database of disease symptoms mapped to possible causes and accepting a list of user symptoms from the user. The diagnostic module (400) may further comprise instructions for generating a first table (410) mapping each user symptom to all possible causes for the user symptom. The first table (410) may be filtered using data from the database of medical records comprising an age, a gender, a weight, and a medical history of the user. The diagnostic module (400) may further comprise instructions for generating a second table displaying all possible causes (450) that share every user symptom, accepting data from the database of medical records (2100), generating a probability table ranking all possible causes (450) based on probability of the presence of each possible cause, and determining a treatment (475) for the possible causes based on the data from the database of medical records (2100). The probability table may be generated for every user symptom. The probability weights may be calculated by dividing the number of symptoms matched between the user symptoms and the symptoms of the possible cause by the total number of user symptoms. In some embodiments, probability weights may be adjusted according to illnesses reported in a radius around the user's location. In some embodiments, the diagnostic module (400) may further comprise instructions for requesting a plurality of tests from the user to better determine the possible cause, and the software platform (600) may further comprise testing equipment for allowing the user to carry out the plurality of tests. The testing equipment may comprise a personal vital signs monitor, a personal EKG, a glucometer, an ultrasound imaging device, a cardiac monitor, and a skin image camera. In some embodiments, the software platform (600) may further comprise a test analyzer module (500) for accepting test results from the user and processing the test results to aid the diagnosis platform in generating the possible cause. For a patient reporting just one symptom, the platform may check the patient's medical history, illnesses reported in the region, and perform more tests to obtain more information to determine the most likely illness. In some embodiments, the database of disease symptoms is stored in a cloud server and accessed by the diagnostic module (400).

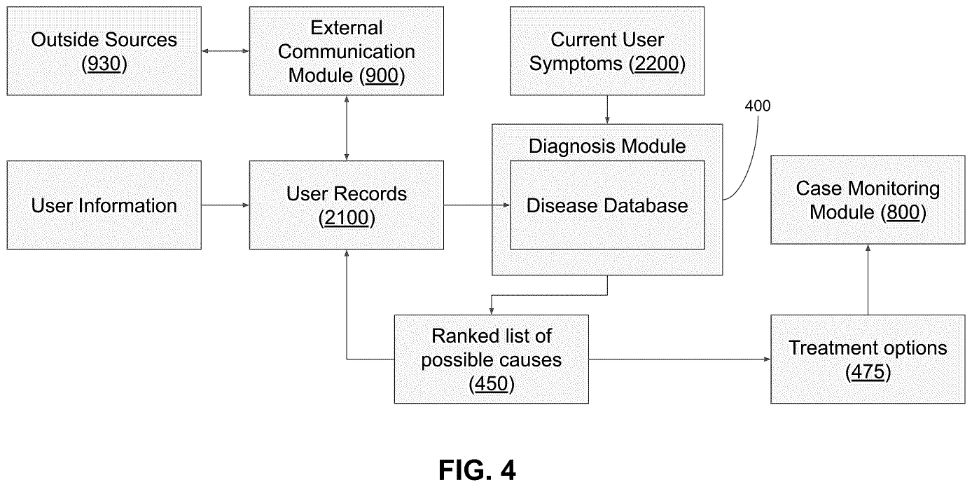

Referring now to FIG. 4, the present invention features a software platform (600) for compiling a user's medical history from a plurality of sources and using the medical history to diagnose a medical issue of a user. The platform may comprise an external communicator module (900) for gathering information pertaining to the user's medical history from a plurality of sources. The external communicator module (900) may comprise a data encryption module (910) having instructions for accepting data from a source, and encrypting the data into a standard format. The data encryption module (910) may be capable of accepting data directly from the user, or a plurality of external medical sources (930). The external communicator module (900) may further comprise a data compression module (920) having instructions for accepting encrypted data from the data encryption module (910), compressing the encrypted data into compressed data, and transmitting the compressed data to an external storage component (9000) or the plurality of external medical sources (930). The platform may further comprise a database of medical records (2100). The database of medical records (2100) may comprise a medical history of the user, a list of pre-existing conditions of the user, and a list of medications currently taken by the user.

The platform may further comprise a diagnostic module (400) having instructions for accessing a database of disease symptoms mapped to possible causes and accepting a list of user symptoms from the user. The diagnostic module (400) may further comprise instructions for generating a first table (410) mapping each user symptom to all possible causes for the user symptom. The first table (410) may be filtered using data from the database of medical records comprising an age, a gender, a weight, and a medical history of the user. The diagnostic module (400) may further comprise instructions for generating a second table displaying all possible causes (450) that share every user symptom, accepting data from the database of medical records (2100), generating a probability table ranking all possible causes (450) based on probability of the presence of each possible cause, and determining a treatment (475) for the possible causes based on the data from the database of medical records (2100). The probability table may be generated for every user symptom. The probability weights may be calculated by dividing the number of symptoms matched between the user symptoms and the symptoms of the possible cause by the total number of user symptoms. In some embodiments, the diagnostic module (400) may further comprise instructions for requesting a plurality of tests from the user to better determine the possible cause, and the software platform (600) may further comprise testing equipment for allowing the user to carry out the plurality of tests. The testing equipment may comprise a personal vital signs monitor, a personal EKG, a glucometer, an ultrasound imaging device, a cardiac monitor, and a skin image camera. In some embodiments, the software platform (600) may further comprise a test analyzer module (500) for accepting test results from the user and processing the test results to aid the diagnosis platform in generating the possible cause. In some embodiments, the database of disease symptoms is stored in a cloud server and accessed by the diagnostic module (400).

The platform may further comprise a case monitoring module (800) having instructions for monitoring the user's health and recovery as the user carries out the treatment (475) determined by the diagnostic module (400), reminding the user to carry out a prescription related to the treatment (475), comparing an expected recovery time to an actual recovery time to determine if a secondary course of action is necessary, adjusting the prescription as the secondary course of action, and adjusting the expected recovery time based on the secondary course of action.

In some embodiments, the software platform (600) may further comprise a treatment module (700) for generating a plurality of treatments for the plurality of possible causes generated by the diagnostic module (400) and allowing the user to select a route of treatment. In some embodiments, the external communicator module (900) may further comprise instructions for ordering the prescription to be delivered to the user. In some embodiments, the software platform (600) may further comprise a nursing services module (1000) for allowing the user to request a registered nurse or licensed health care provider to aid the user with medical issues, testing, or treatment. In some embodiments, the software platform (600) may further comprise a dietician (1100) module for keeping track of the user's dietary habits as data to be added to the database of medical records (2100) and for aiding the user in maintaining a healthy diet in accordance with the treatment determined by the platform. In some embodiments, the software platform (600) may further comprise a central research module (1200) for communicating with a database of data recorded by a plurality of instances of the platform to transmit and receive test results, diagnoses, and recovery times to improve calculations and overall performance of the platform. The nursing services module (1000) may comprise instructions for allowing a volunteer to register onto a list of nurses through the software platform (600) or transmitting registration data to the virtual physician (600) through the external communicator module (900). The nursing services module (1000) may further comprise instructions for contacting a nurse from the list of nurses for an in-home visit.

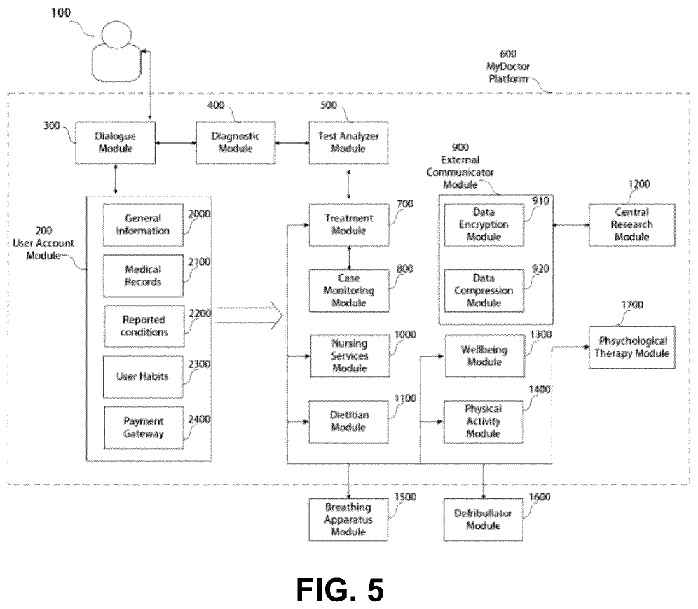

The present invention features the software platform (600) that could be uploaded on a cloud where various modules that could be uploaded and integrated and peripherals (personal diagnostic devices) could also be connected to the platform as well. The software platform (600) could be accessed through a smartphone application or a standalone device linked to the internet. FIG. 5 shows a diagram that demonstrates the general scheme of the invention.

The modules of the software platform (600) may be integrated using a suitable programming language such as Python as a back-end or similar language and friendlier language such as PHP as front end. The software platform (600) may have accessibility to communicate with various relevant parties such as medical centers including clinics, hospitals, pharmacies, Labs and emergency centers (e.g. 911). Various modules that are connected and integrated are uploaded on the software platform (600).

The modules of the software platform (600) may be executed on one or more computing devices. A computing device may be a standalone device (e.g. a personal diagnostic device), a personal computing device, a cloud computing device, a mobile computing device, Each computing device may comprise at least a processor capable of executing computer-readable instructions, and a memory component comprising the plurality of modules, each module comprising a plurality of computer-readable instructions capable of being executed by the processor. Each computing device may further comprise a communication component allowing a computing device to communicate with other computing devices. The communication component may comprise a wired and/or wireless Internet connection component, a Bluetooth connection component, etc. In some embodiments, the modules of the present invention may be split across multiple computing devices. For example, the front end processing (e.g. the Dialogue Component (300)) may be carried out on a personal computing device and/or a mobile computing device and the back end processing (all other modules) may be carried out on a cloud computing device. Note that any configuration of modules executed on multiple separate computing devices is a possible embodiment of the present invention. In some embodiments, most of the processing shall be conducted in the central cloud solution. Limited edge processing will take place for very limited activities such as reading from the devices (e.g., thermometer) .

The present invention features a virtual physician platform comprising an autonomous software system configured to emulate a healthcare provider, the system comprising a hardware processor and non-transitory computer-readable medium storing computer-readable instructions. The non-transitory computer-readable medium may further store a user interface module configured to receive verbal/textual input from a user and to provide verbal/textual responses, utilizing natural language processing techniques. The non-transitory computer-readable medium may further store a medical data management module configured to store and retrieve user data, including personal information, medical records, reported conditions, and user habits, received from external sources and converted into a standardized format. The non-transitory computer-readable medium may further store a diagnostic module configured to access a database of disease symptoms mapped to possible conditions, accept symptom data from the user interface and test results from an external test analyzer, generate and analyze probabilistic models correlating symptoms with potential causes, and output diagnostic suggestions based on these models.

The non-transitory computer-readable medium may further store a treatment module configured to determine a treatment plan based on diagnostic outputs and medical records, generate prescriptions and recommendations, and communicate prescription and recommendation information to an external pharmacy network. The non-transitory computer-readable medium may further store a monitoring module configured to continually assess user health status during treatment, notify the user about medication schedules and health reminders, and adjust treatment plans based on recovery data and secondary assessments. The non-transitory computer-readable medium may further store an external communicator module configured to securely transmit and receive encrypted medical and testing data with external sources and compress/decompress data for storage or transfer. The non-transitory computer-readable medium may further store auxiliary modules including dietary management, physical activity guidance, respiratory support, cardiac emergency response, and psychological health assessment, each configured to collect relevant data and provide personalized recommendations. The platform may integrate these modules into a cohesive system that dynamically interacts to provide autonomous medical guidance, diagnostics, and treatment recommendations based on user data and medical knowledge databases.

In some embodiments, the external communicator module (900) may be further configured to communicate with a hospital to receive medical records of the user and scheduling information for appointments, and transmit the user data and appointment requests to the hospital. The external communicator module (900) may be further configured to communicate with a medical laboratory to receive medical records, scheduling information, and lab results, and transmit the user data and appointment requests to the laboratory. The external communicator module (900) may be further configured to communicate with a clinic to receive medical records and scheduling information for appointments, and transmit the user data and appointment requests to the clinic. The external communicator module (900) may be further configured to communicate with an emergency center to transmit user location data and request emergency services such as an ambulance. The external communicator module (900) may be further configured to communicate with a pharmacy to receive medical records and pricing information for prescriptions, and transmit user data, prescription details, and delivery instructions to the pharmacy.

The present invention features a virtual physician platform comprising an autonomous software system (600), configured to facilitate diagnostic testing and treatment recommendations for a user. The platform may comprise a containment and execution environment for the virtual physician software. The virtual physician software may comprise a medical records database (2100) that comprises data elements including a medical history of the user, a list of pre-existing conditions, and a current medications list. Data received in the database may be collected from a plurality of external medical sources (930) and converted into a standardized format upon receipt, enabling interoperability and consistent data processing.

The platform may further comprise a diagnostic module (400), comprising instructions stored and executable on a processor to perform a plurality of functions. The functions may comprise accessing a disease database that maps disease symptoms to potential causes. The functions may further comprise accepting a list of user-reported symptoms. The functions may further comprise generating a first table (410) that maps each symptom to all possible causes. The functions may further comprise generating a second table (450) that identifies causes sharing all user symptoms. The functions may further comprise incorporating medical records data received from the external sources (930) into diagnostic processing. The functions may further comprise generating a probability ranking of causes based on symptoms and medical history using a Random Forest Classifier algorithm. The functions may further comprise requesting specific diagnostic tests from the user to refine cause identification. The functions may further comprise accepting test results from external testing equipment or user-provided data. The functions may further comprise updating the cause probability ranking based on test results. The functions may further comprise autonomously generating a treatment plan for the most probable cause(s) based on the combined data, including medical history, symptoms, and test results, utilizing the Random Forest Classifier algorithm. Each module of the software system may be configured to transmit data to other modules, enabling integrated processing and diagnostic decision-making, thereby providing a comprehensive autonomous medical diagnosis and treatment suggestion system. The platform may further comprise disease database that maps symptoms to possible diseases.

The testing equipment may comprise a hardware system. The hardware system may comprise a personal vital sign monitor configured to measure and transmit physiological parameters such as heart rate, blood pressure, and oxygen saturation. The hardware system may further comprise a personal electrocardiogram (EKG) device capable of recording and transmitting cardiac electrical activity. The hardware system may further comprise a glucometer configured to measure and transmit blood glucose levels. The hardware system may further comprise an ultrasound imaging device capable of capturing and transmitting real-time ultrasound images of internal body structures. The hardware system may further comprise a cardiac monitor adapted to continuously monitor cardiac function and transmit relevant data. The hardware system may further comprise a skin imaging camera designed to capture high-resolution images of the user's skin for diagnostic analysis. Each of the above devices may be configured to connect to the digital platform for real-time data transmission and integrated analysis.

The platform may further comprise a test analyzer module (500), configured to receive raw test data transmitted from testing equipment associated with the user, process the raw test data using dedicated hardware and algorithmic components to generate a set of test results, and provide the generated test results to the diagnosis platform to facilitate identifying potential causes of the user's medical condition. The test analyzer module may be implemented as a hardware-accelerated processing unit or includes dedicated hardware circuitry configured to perform the test data processing, thereby enabling real-time or near-real-time diagnostic support.

The database of disease symptoms may be stored on a cloud-based server, and the diagnostic module (400) may be configured to electronically access and retrieve data from the cloud server via a secure network connection for use in diagnostic processing. The system's diagnostic module may be configured to generate a separate probability table for each user-reported symptom. Each probability table may quantify the likelihood of various possible causes based on the symptom data and associated medical databases. The system's diagnostic module may be configured to calculate probability weights for each possible cause by determining a ratio by dividing the number of user-reported symptoms that match the symptoms associated with each possible cause by the total number of user-reported symptoms, and further narrowing down the list of potential causes through additional diagnostic tests and interactive communications with the user to refine and identify the most probable cause. The calculation of probability weights and the iterative narrowing process may be performed using algorithmic operations executed by hardware-implemented processing components. The first table (410), which maps symptoms to potential causes, may be dynamically filtered using relevant patient data including age, gender, weight, and medical history retrieved from the database of medical records, thereby refining the diagnostic results based on user-specific information.

The present invention features a virtual physician (MyDoctor) autonomous platform (600) configured to compile a comprehensive medical history of a user from multiple sources and utilize this information to diagnose a medical issue. The platform may comprise a means for containing and executing virtual physician software. The software may comprise an external communicator module (900) configured to collect user medical information from multiple sources. The external communicator module (900) may comprise a data encryption module (910) executed by hardware-based components, configured to accept data from local or remote sources and convert the data into a standardized format for interoperability. The external communicator module (900) may further comprise a data compression module (920) designed to compress the standardized data and transmit it securely to external storage (9000) or medical sources (930). The software may further comprise a centralized database (2100) configured to store received medical data, including user's medical history, pre-existing conditions, and current medications. Data from external medical sources (930) may be reformatted into a standard structure upon receipt.

The software may further comprise a diagnostic module (400), comprising instructions stored in hardware or firmware. The diagnostic module (400) may be configured to access a disease database mapping symptoms to potential causes. accept a list of user-reported symptoms. The diagnostic module (400) may be further configured to generate a first cause-symptom mapping table (410) based on user symptoms. The diagnostic module (400) may be further configured to generate a second cause list (450) sharing all user symptoms. The diagnostic module (400) may be further configured to incorporate medical history data from the database (2100). The diagnostic module (400) may be further configured to compute a probability ranking of causes based on symptom match and available data using an algorithm such as a Random Forest classifier. The diagnostic module (400) may be further configured to request additional diagnostic tests from the user to refine cause determination. The diagnostic module (400) may be further configured to accept test results from testing equipment or user input. The diagnostic module (400) may be further configured to update the cause probability ranking based on test outcomes. The diagnostic module (400) may be further configured to autonomously generate a treatment plan based on combined data, symptoms, and test results.

The software may further comprise a case monitoring module (800), comprising instructions stored in hardware or firmware. The case monitoring module (800) may be configured to monitor the user's health status and recovery during treatment. The case monitoring module (800) may be further configured to remind the user to adhere to prescriptions. The case monitoring module (800) may be further configured to compare expected versus actual recovery times to identify if secondary actions are needed. The case monitoring module (800) may be further configured to adjust prescriptions or treatment as necessary. The case monitoring module (800) may be further configured to update expected recovery times based on secondary interventions. Each module may be configured for data transmission and communication with other modules within the platform to enable integrated, automated diagnosis and treatment management. The platform may further comprise a disease database correlating symptoms to possible diseases. The platform may further comprise an external storage component (9000) configured to store system data, including medical records, test results, and generated reports.

The diagnostic module (400) may comprise instructions executable by hardware to generate and transmit test requests to the user or testing apparatus, based on the current diagnostic assessment, to obtain additional test data for refining the diagnosis, wherein the process of requesting tests involves automated, data-driven decision-making performed by hardware-implemented algorithms. The platform may further comprise hardware-based testing equipment configured to securely collect physiological and diagnostic data from the user, perform a plurality of medical tests (such as measuring vital signs, capturing images, or other diagnostic procedures), transmit the test data to the platform's diagnostic module for analysis, and enable real-time or near-real-time processing for diagnosis and treatment planning.

The testing equipment may comprise a suite of hardware devices including a personal vital sign monitor configured to measure blood pressure, heart rate, and oxygen saturation, a personal electrocardiogram (EKG) device capable of recording and transmitting cardiac electrical signals, a glucometer for measuring blood glucose levels, an ultrasound imaging device configured to capture real-time internal body images, a DNA sampling device adapted to collect genetic material for analysis, a cardiac monitor for continuous cardiac activity tracking, and a skin imaging camera capable of capturing high-resolution images of the user's skin for dermatological assessment. Each device may be configured as a hardware component that interfaces with the platform to transmit diagnostic data for processing and analysis.

The platform may further comprise a hardware-implemented test analyzer module (500), configured to receive raw test data transmitted from testing equipment, process the raw data using hardware-accelerated algorithms or dedicated circuitry to generate a structured set of test results, and store and transmit these test results to the diagnostic module to facilitate the identification of possible causes. The processing of test data may involve concrete, algorithmic operations executed by hardware components, thereby providing tangible technological functionality. The platform may further comprise a hardware-implemented treatment module (700) configured to autonomously generate multiple treatment options for each possible cause identified by the diagnostic module (400) using algorithmic processing executed by hardware circuitry, present the generated treatment options to the user through a user interface, enabling the user to select a preferred treatment route, and update and refine the treatment plan based on user selection and ongoing monitoring, with all processing performed by tangible hardware components.

The external communicator module (900) may be configured as hardware-implemented instructions, including functionality to securely generate and transmit an electronic prescription order to a designated pharmacy or delivery service, enabling the prescribed medication to be delivered directly to the user. The platform may further comprise a dietitian module (1100), implemented as hardware-encoded instructions, configured to collect and digitally record the user's dietary habits and nutritional intake, integrate the collected dietary data into the user's electronic medical record (2100), analyze the dietary data using algorithmic processing to provide personalized recommendations for maintaining a healthy diet, weight management, and avoiding food-drug or food-condition interactions based on the diagnosis and treatment plan established by the platform, and present dietary guidance and recommendations to the user via a user interface.

The present invention features an autonomous medical management system accessible via a smartphone application or web platform. In some embodiments, the system may comprise a processor. The system may further comprise a non-transitory computer-readable storage medium storing instructions. When executed by the processor, the computer-readable instructions may cause the system to receive user input via an audio interface and/or a typed input interface. The instructions may further cause the system to convert spoken input into digital text using hardware speech recognition modules. The instructions may further cause the system to analyze the input employing hardware-accelerated natural language processing algorithms to extract symptoms, commands, or requests. The instructions may further cause the system to communicate with external medical data sources through hardware network interfaces, including encrypting and compressing data using hardware modules. The instructions may further cause the system to retrieve, compile, and store medical records, sensor data, and interaction logs.

The instructions may further cause the system to analyze the combined medical data and user input with hardware-implemented diagnostic algorithms to identify potential health conditions. The instructions may further cause the system to monitor treatment progress using physiological sensors or follow-up interactions supported by hardware modules. The instructions may further cause the system to generate diagnoses and treatment recommendations based on the analysis. The instructions may further cause the system to transmit prescriptions to pharmacies or healthcare providers via network interfaces. The instructions may further cause the system to initiate emergency calls, transmitting user location and health data to emergency services. The instructions may further cause the system to schedule or modify healthcare appointments via hardware communication modules. The instructions may further cause the system to provide alerts, reminders, and user feedback through tangible output components such as speakers and screens.

In some embodiments, the user input may comprise spoken commands processed through hardware speech recognition modules, typed commands entered via touchscreens or keyboards are analyzed with hardware-accelerated NLP algorithms, or a combination thereof. In some embodiments, the system may further comprise medical sensors physically connected to the platform, configured to collect physiological data such as vital signs, electrocardiogram (ECG), ultrasound images, or laboratory test results, hardware modules for analyzing this sensor data to evaluate health status and treatment efficacy, or a combination thereof. In some embodiments, the platform may be implemented as a smartphone application or web-based platform accessible through a network. The application or platform may operate on tangible hardware modules capable of executing input/output functions, data processing, and communication. In some embodiments, functions such as appointment scheduling, prescription ordering, emergency calling, and health status monitoring are performed via hardware modules capable of secure data transmission over a network interface integrated into the device.

The present invention features a method performed by a hardware-implemented platform accessible via a smartphone application or web interface. The method may comprise capturing user input through verbal interaction supported by hardware speech recognition modules, or through typed commands entered via hardware input interfaces. The method may further comprise analyzing the input using hardware-accelerated natural language processing algorithms to identify symptoms, requests, or commands. The method may further comprise retrieving medical data from external sources via hardware communication interfaces. The method may further comprise analyzing the combined data using hardware algorithms to diagnose potential health conditions. The method may further comprise generating treatment plans and prescriptions. The method may further comprise executing healthcare management functions such as transmitting prescriptions, scheduling or rescheduling appointments, and calling emergency services through hardware communication modules. The method may further comprise monitoring health status via physiological sensors and follow-up interactions enabled by hardware modules. The method may further comprise updating diagnosis and treatment recommendations dynamically based on ongoing physiological and interaction data.

In some embodiments, data collection, analysis, and task execution may be performed through hardware modules integrated into the device. The platform may be capable of autonomously managing prescriptions, emergency calls, and appointment scheduling via hardware-enabled secure communication.

The present invention features a tangible hardware system. The system may comprise one or more input/output interfaces including microphones, touchscreens, and speakers. The system may further comprise one or more hardware modules for speech recognition and natural language processing. The system may further comprise communication hardware for secure data exchange with external medical facilities, pharmacies, and emergency services. The system may further comprise physiological sensors for real-time data collection. The system may further comprise processing modules executing algorithms to analyze medical data and user interactions, supporting autonomous health diagnosis, treatment, and management.

In some embodiments, the platform may be hosted on a remote server, and the modules and functions are executed as hardware-accelerated processes on tangible infrastructure accessible via the internet or local network. In some embodiments, the system may further comprise hardware modules configured to facilitate scheduling and management of healthcare appointments, transmit prescriptions with pricing and delivery options, initiate emergency calls with user location and vital sign data, and process natural language commands received via voice or text interfaces for autonomous healthcare management.

In some embodiments, the platform of the present invention may comprise a data encryption module (910) as a component of the external communicator module (900). The data encryption module (910) may be configured to encrypt the data received from the plurality of sources into a format that cannot be interpreted or intercepted as a result of data breaches, cyberattacks, etc. This is a benefit of the present invention, as one of ordinary skill in the art may expect that the implementation of a centralized hub for sensitive user medical information would be too much of a security concern to be viable. The encryption and compression of the data into a format that can be securely transferred and efficiently accessed and used in the disease diagnosis and treatment determination of the present invention could only be achieved by a computing device such as that implemented in the presently claimed invention.

In some embodiments, the functionality of the present invention may be implemented entirely on a personal computing device, such as a desktop computer, a laptop computer, a smartphone, or a combination thereof. In some embodiments, the functionality of the present invention may be implemented nearly entirely on a cloud computing system in communication with the user's personal device (e.g., through a website, through a smartphone app, etc.). In these embodiments, the personal device may only be used for interface, user input, and display purposes. In some embodiments, any combination and assortment of functions may be divided between the personal device and any number of cloud computing devices.

In some embodiments, the user input accepted by the present invention may comprise audio input from the user, received through a microphone component coupled to the personal computing device. The audio input may be processed by a natural language processing algorithm, configured to translate the audio input into strings usable by software platform. In some embodiments, the user input accepted by the present invention may comprise typed input, received through a keyboard component (e.g. physical or touchscreen) coupled to the personal computing device. In some embodiments, the input received from the user may be processed by a natural language processing algorithm/speech recognition module to extract keywords useful for the functionality of the software platform. The natural language processing algorithms implemented in the present invention may be well-known algorithms that function as well-described in the art.

In some embodiments, the present invention may implement input from physiological sensors communicatively coupled to the personal computing devices, the cloud computing devices, or a combination thereof to identify symptoms in the user (e.g., measuring temperature, heart rate, blood pressure, etc.), track the progress of treatment on the user, or a combination thereof. The physiological sensors may comprise cameras, microphones, or a combination thereof to analyze facial expressions, emotions, and postures, as well as the tone of voice.

In some embodiments, the present invention may be configured to generate treatments for the diagnosed diseases in the user in the form of prescriptions for medication. In some embodiments, the present invention may have the functionality (e.g., through the external communicator module) to transmit the prescriptions to pharmacies that can fulfill them. In some embodiments, the User Account Module may be configured to accept and store the user's location. The user's location information may be used to identify a viable pharmacy in proximity to the user that can fulfill the prescription. In some embodiments, the prescription information that is transferred to the pharmacy may be encrypted and/or compressed as it is transferred from the present invention to the pharmacy.

In some embodiments, the present invention may implement one or more machine learning models throughout the modules of the software platform. The machine learning models may be used for diagnosis of one or more diseases in the patient based on the user symptoms and medical history, determining the treatment of the one or more diseases in the patient based on the one or more diseases and the user medical history, tracking a progress of treatment in the user based on the one or more diseases, the treatment, and the medical history, or a combination thereof. Additionally, other Artificial Intelligence (AI)-based models may be used for generating prescriptions, Natural Language Processing, identifying the best pharmacy to transmit the prescriptions, encryption, security measures, or any other functionality of the software platform.

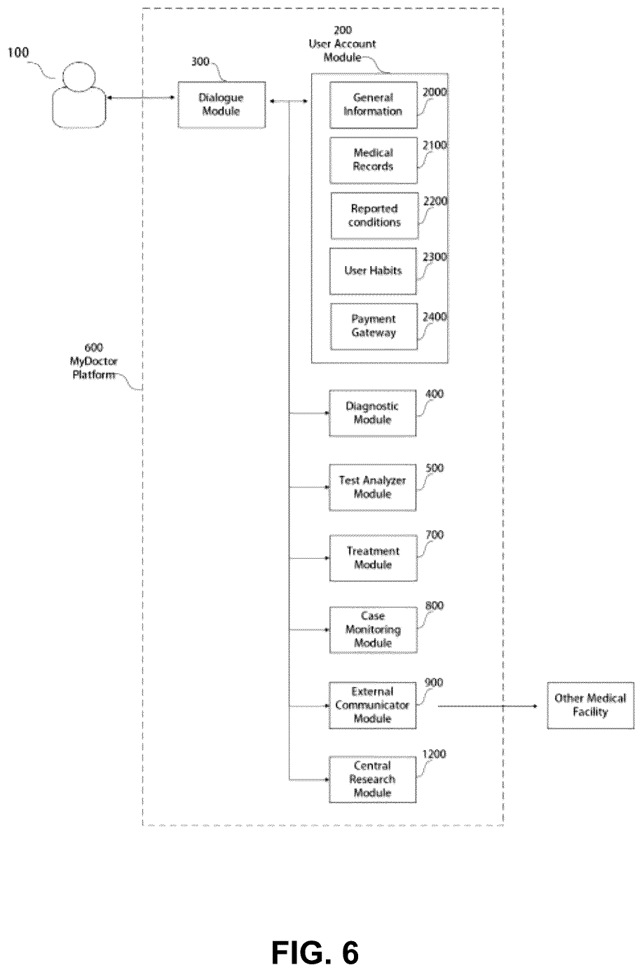

FIG. 6 shows the layout of the User Account. The User Account Module may be connected to all other modules where the data may be exchanged to and from the Patient account. Each user (100) should have an account where information about the user (100) should be entered. The required information for registration could be entered verbally or manually. If the request is verbal, the system may convert it to text using Speech Recognition algorithms (e.g. Linux Speech Recognition Software-Open Source) through the Dialogue Module (300). The user (100) should turn on the application and if it is his/her first time, a smart form has to be filled. The system may ask the user (100) questions (audibly) to fill out the form such as name, age, gender, etc. Once the smart form is filled and all mandatory questions are answered and requirements filled, the user (100) may have an active account.

Another option to expedite the registration process is to link to the National ID Number (e.g. Social Security), once the National ID is inserted the system may fill out the general information such as name, age, personal picture and fingerprint. Therefore, the user (100) may only fill out or authorize to retrieve the historical medical information and payment information for billing. In an emergency, authorized personnel or emergency response team could log into the user (100) account via voice/user fingerprint/user facial recognition where this feature is important to enter into the User Account Module (200) when the user (100) is unconscious. Each time, the user (100) interacts with the system the tone of voice and facial expressions may be recorded. As the user (100) keeps using the system, the database of the tone of voice and facial expressions could be processed using Machine learning to determine the user (100) sentiments and feelings.

Voice tone analysis is critical in determining the User (100) mood, stress, pain, anxiety, and depression. As more data is collected on the user (100), the software platform (600) can immediately determine the urgency of the user (100) request. The User Account Module (200) may consist of many features and options including:
1. General Information (2000)
2. Medical Records (2100)
3. Reported conditions (2200)
4. User Habits (2300)
5. Payment Gateway (2400)

As part of the User Account Module (200), the General Information (2000) of the user (100) may contain information to identify the user (100). This information could also be shared with other medical providers to identify the user (100) and to obtain the user (100) medical records.

Medical records (2100) from the user (100) may be retained to provide better and expedited diagnosis and to assist with the Central Research Module (1200). At the same time, the User (100) external medical records can be uploaded to the user medical records (2100) through connecting the Electronic Health Record (EHR) or Hospital Information Support System (HISS) to the software platform (600). The Medical Records (2100) may be updated for any cases of illness, stress, discomfort, allergies to track the health of the user (100) with time. The Medical Record (2100) may communicate with Case Monitoring Module (800) so that the progress of the treatment can be recorded. Tests Analyzer Module (500) is also linked to the Medical Record to log the results of tests and examinations. External Communicator Module (900) is linked to the Medical Record to feed the Medical Records from the external facilities such as labs and physician input. The central research module (1200) may comprise instructions for storing disease data and recovery data from a plurality of users in a research database, and analyzing the disease data and the recovery data from the research database to aid in diagnosis of a disease and recovery time of a treatment method.

At the same time, the External Communicator Module (900) may provide outside medical facilities such as physicians with access to the patient's medical records (2100). The Medical Records may also communicate to the Diagnostic Module (400) to support the diagnosis process and to the Treatment Module (700) to record the prescribed treatment. It is also linked through a one-way communication channel to feed Central Research Module (1200).

For underage (e.g. infants) or those who do not have the capacity to use the software platform (600), for any reason, a trustee could interact with the software platform (600) on behalf of the user (100).

With more interaction and usage from the user (100), the Reported conditions (2200) of the user (100) may be kept and shared with the Central Research Module (1200). This information is critical in M.L. in predicting illnesses and tracking the user (100) health as they get older. This information is useful to share with medical insurance companies to determine high-risk patients, high-risk ages and to issue preventive care before a serious illness might occur.

The User Habits (2300) are stored and shared with other modules in the software platform (600). This information is used with the Dietitian Module (1100), Central Research Module (1200), Wellbeing Module (1300), Physical Activity Module (1400), and Psychological Therapy Module (1700). The information collected could be daily exercise, nutrition, favorite foods, sleep habits, and quality of sleep. The Payment Gateway (2400) is used to work with the External Communicator Module (900) to make payments to Pharmacies, doctor's offices, and to purchase add-ons to Modules or subscription services.

The Payment Gateway (2400) may have two options, meeting all PCI DSS requirements for the user (100) to make payment as payment is needed at the time of service. Or to have the User (100) payment information on file during the creation of the User Account, where PCI DSS requirements may be met at the back office.

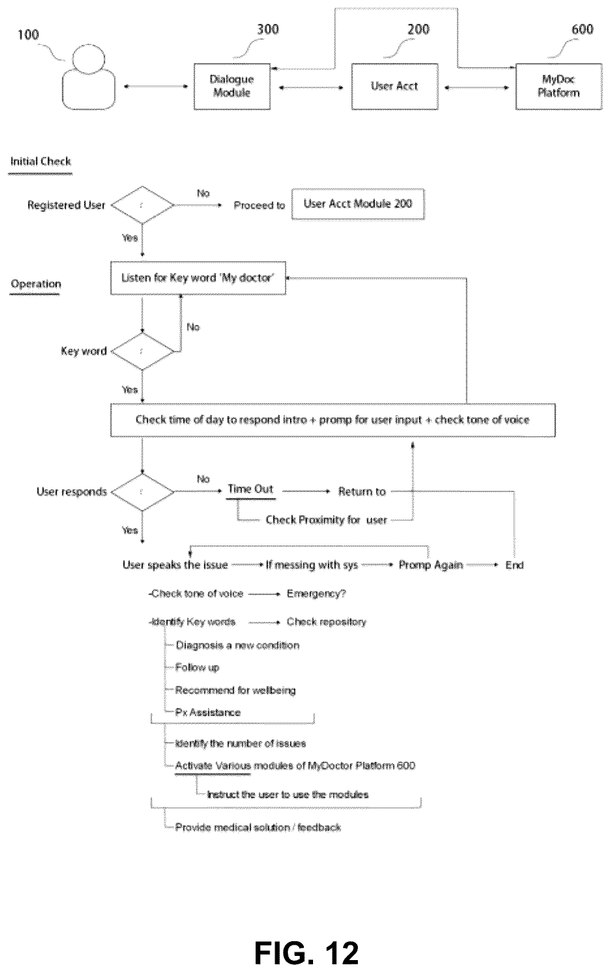

FIG. 12 shows the layout of the Dialogue Module 300. The Dialogue Module (300) may be connected to other modules where the data may be exchanged to and from the user (100) and the logic flow process. This module may be the interface between the user (100) through the User Account (200) and the software platform (600). The objective of this module is to convert the text to verbal conversion and vice versa. This module may employ speech recognition and Natural Language Processing to communicate with the User (100). The system may have predefined phrases with multiple versions for the same phrase. For example, whenever the User (100) calls the system “MyDoctor” the system may reply and ask the user (100) “how can I serve you”. The user (100) could simply say anything. However, the system may focus on medical conditions using algorithms such as BERT as a tool to perform NLP. The dialogue between the system and the user (100) may take a format of question and answer. When the user 100 says something relevant to his physical or even psychological condition the system may interact and ask questions to diagnose the condition as a first step. To demonstrate the possible dialogue, the following scenario could take place:

The System: “Good Morning Mr. David, I am your physician Dr. Mazen; how can I help you?” (The greetings may depend on the time of the day. Other welcoming statements could be prompted to give the system a more natural feel. In case, the user (100) is following up with the system after reporting a medical condition the greetings statement may be different. It could be “how do you feel now? A predefined protocol may be loaded to manage the flow of the communication)

The user (100) has to answer the system. The following scenarios could take place:
The User (100) keeps silent
The User (100) could keep talking in a subject irrelevant to his/her physical or psychological condition
The User (100) states his/her problem but it could be not in a clear manner.

For each scenario, a certain course of action should take place. The following is the typical system's behavior for each one of the above scenarios:
1. If the user (100) keeps silent the system after 10 seconds may rephrase the greetings and the question. If the user (100) keeps silent the system may be on hold mode. This behavior could be different in the case depending on the situation. Adding a sensor (infrared or camera) could also help the system to know if the User (100) is within the vicinity of the device. This may be doable if the standalone version of the proposed invention is adopted.
2. If the user (100) starts saying nonsense words the (100) system may state that “I am here to provide you with a medical care service. I can only help you if you need medical help”. The system may utilize cameras and other sensors to determine if the user (100) is in distress and call for an emergency response team as necessary. If the user (100) keeps playing with the system the system may timeout with “Sorry, I cannot help you.”
3. If the user (100) says anything relevant to a medical issue the system may recognize the words of attention (feel/headache/medicine etc.) which could be one out of many defined in the repository dictionaries with synonyms.

The system may lead the conversation based on the initial input of the user (100). The conversation between the user (100) and the system may search a predefined repository to determine what task to perform and to focus on one of the following tasks:
Diagnosis a new condition
Follow up of existing condition
Psychological Therapy
Recommendation for wellbeing
Medical assistance such as ordering medicine from a pharmacy or making an appointment with a specialist

By using NLP this module may specify the task and relevant modules may be triggered with its appropriate flow of the dialogue. If the user (100) mixes up more than one topic. The system may segregate the issues and address each topic separately. For example, if the User (100) says “I have a headache and I need to order a refill for my diabetic medicine.” In this case, the system may address the headache separately from ordering the refill but before it does so the system may verify that there is no association between both requests. Structuring the conversation by the system is important to achieve a certain task.

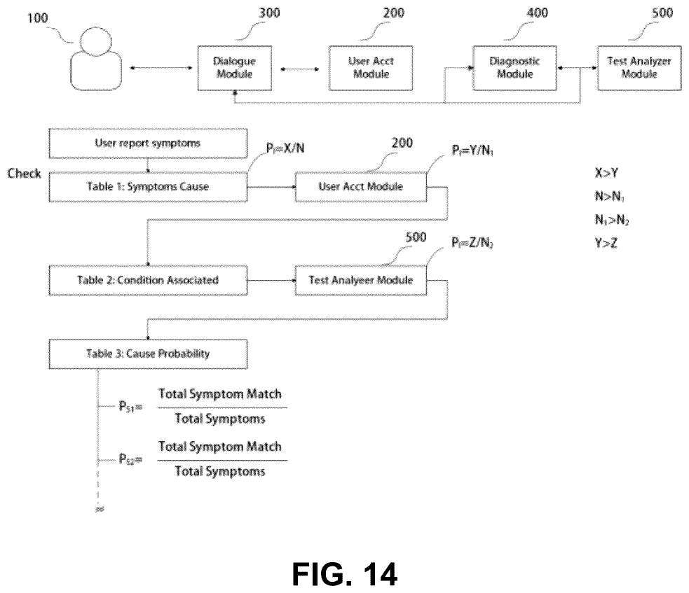

FIG. 14 shows the layout of the Diagnostic Module (400). The Diagnostic Module (400) is a repository of all human being known diseases with their signs and symptoms and the tests that are necessary to confirm the condition along with algorithms employing the deep learning and Artificial Neural Network concept.

The Diagnostic Module (400) is communicating with Dialogue Module (300) to receive the symptoms from the patient and to provide the patient with the necessary tests and examinations to be performed. Medical Record Modules also communicate with the Diagnostic Module (400) as input to the search algorithm. Test Analyzer Modules (500) Module provides the results of the various tests to be used in the diagnosis process. This module may be triggered once the task requested by the User (100) is determined in the Dialogue Module (300) to be a request to diagnose the User (100) medical conditions. The keywords and symptoms that the user (100) mentions to the Dialogue Module (300) may be used to search in the Diagnosis tables. The Module may ask the user questions regarding the symptoms. The more variables (symptoms) provided the better search could be conducted. The input from the vital signs device may also be used as a search variable. To be able to conduct the diagnosis, certain input may be essential to isolate the condition. To narrow the search, three tables may be searched initially. The first table is symptoms cause table where all symptoms are tabulated and under each symptom, all possible related causes (diseases) are tabulated. The list of the symptoms (number of columns) could be more than 84 symptoms. The symptom could be a noun (Fever) or adverb (e.g. cannot breathe). The system may conduct its initial filtering. For example, if the reported symptom is only fever, the system may focus on all possible causes for fever and run a sanity check for each possible cause. Some of the causes could be a group of diseases such as infection, dehydration, medications currently taken, or heatstroke. In this case, another table is built for this group. The criteria for sanity check may be age, gender, weight, severity of the symptoms, and user (100) medical records (2100). For each cause (disease) some factors may affect the probability of the cause. Initially, all possible causes may have equal probability so if there are four possible causes the probability for each one of them is 25%. When the system conducts the filtering for each cause certain predefined factors may affect the probability.

For example, if the patient is not an infant the teething as a cause for fever may have 0% probability. Teething as a possible cause has been excluded with the assumption that the User (100) is an adult based on the general information. Similarly, if the season is winter time the probability of heat stroke is low and could be 0%. By this, the system may narrow the initial search.

The second table is condition-associated symptoms. For example, if the patient only reported having a fever as a symptom, all possible causes may be tabulated. The second table may show all causes for fever and the symptoms of each condition associated with fever. It can be observed that headache is a common symptom between two causes namely: heat stroke and inflammation. Therefore, the system might test these two possible causes by asking the user (100) if they have a headache. If the response is negative this may normalize the weight of inflammation and heat stroke and if the response was positive this should add a higher weight to the probability of heat stock and inflammation over other possible causes (infection).

The third table is the cause probability weight table where the symptoms of each possible cause are ranked based on the probability since we cannot assume that all symptoms bear the same weight. In some cases, there may be a predefined probability weight for each symptom. For example, each symptom of infection has its own weight. Some symptoms are primary and should have a higher probability weight such as fever while secondary symptoms such as digestive upset should bear lower weight. The weight of the symptoms could also be influenced by various factors. Age, gender, severity of the symptom, the duration of the symptoms and user (100) medical records (2100) and history may affect the weight to filer the symptoms cause table, similarly, if the weight of the patient is reasonable obesity as a cause for sweating is going to have 0% probability. The symptoms probability weight table may be created for each reported symptom.

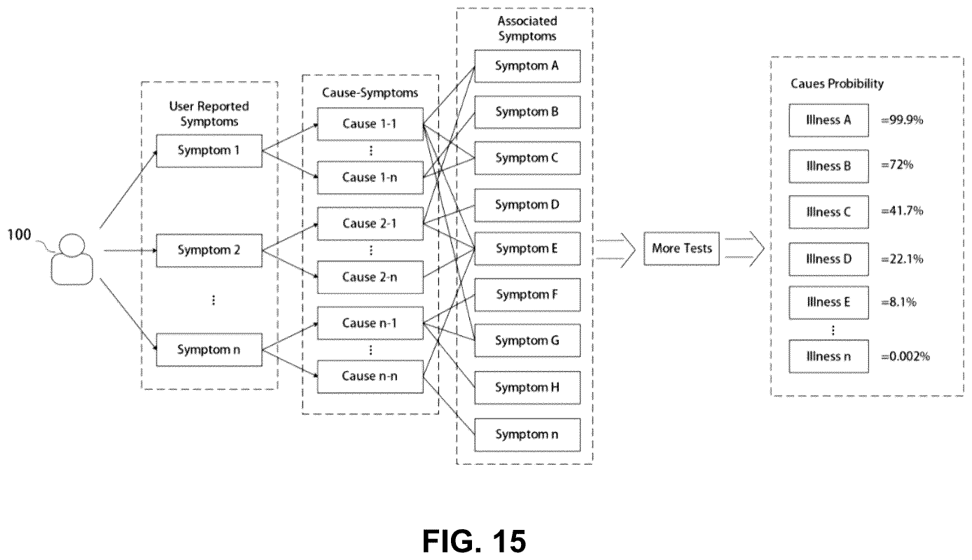

FIG. 15 illustrates the above filtering and probability analysis to narrow the possibilities and determine the next step in the diagnostic process. Based on the probability analysis the system may either ask the patient more questions to narrow the possibilities furthermore or the system might request from the patient to do some tests if the patient couldn't provide additional symptoms. Some symptoms are purely descriptive such as pain or sweating which could be severe or mild (descriptive) while others could be measured such as fever (discreet). For those that could be measured the system may request the patient to do the appropriate exams. The input of the exams may be used to narrow the diagnostic tables furthermore. The medical exams could be certain lab exams or x-rays. The input of those exams should be fed to the system in the patient record. The Diagnostic Module (400) may read the input from the User (100) medical records (2100) to conduct the diagnosis. User (100) may be requested to perform additional tests to enhance the probability and diagnostic. The Random Forest Classifier could perform the diagnosis which is one of the functions under R programming language. In addition, the Diagnostic Module (400) may have a table of all tests and procedures to be used to verify any possible condition (disease). The table lists all tests and procedures and for what condition should be requested. For example, if the patient has some or all of the following symptoms:
Increased thirst
Frequent urination
Increased hunger
Unintended weight loss
Fatigue
Blurred vision
Slow-healing sores
Frequent infections
Areas of darkened skin, usually in the armpits and neck

Classifier Algorithms, in general, provide an autonomous output based on an input. Once the data points are available, the algorithm will generate the output. Once the algorithm is set, it will work autonomously without a need for human intervention. It is worth mentioning that the “Random Forest Classifier Algorithm” is only one example. As an analogy, facial recognition authorisms are sort of classifiers that are able to determine a person Autonomously.

In some embodiments, the software platform of the present invention may be configured to autonomously determine a treatment by executing a Random Forest Classifier algorithm. A Random Forest Classifier algorithm is carried out by setting an initial decision tree based on user input and known data. A decision tree is a graphical representation of different options for solving a problem and shows how different factors are related. It has a hierarchical tree structure starting with one main question at the top called a node which further branches out into different possible outcomes. More decision trees are added to the “forest” as additional information from different instances of user input (e.g., a different user's input, the same user's input at a different time). This additional information is further added to existing decision trees to add complexity and increase accuracy. The Random Forest Classifier algorithm accepts input from the user and runs it through a random subset of the multiple decision trees, each one weighted according to the input, and combines the output from the trees for autonomous determination of an accurate result.

Random Forest and other algorithms such as Gradient Boosting Machines (GBM), Support Vector Machines (SVM), Neural Networks, and others are primarily used for classification tasks. The Random Forest algorithm builds multiple decision trees during training and outputs the mode of the classes for classification or the average prediction for regression across all trees. Besides Random Forest, various other classifiers can be employed in diagnostic applications. Other classifiers encompass, but are not limited to, Gradient Boosting Machines (GBM), Support Vector Machines (SVM), and Neural Networks. These classifiers may be used for diagnosis, treatment determination, or any other process of the system of the present invention.

The random forest example shows that the software platform is autonomous in decision making, self-learning, and improving. The initial decision tree is set and as the data points grow (more user information and more medical illness information) more decision trees can be added or making the existing decision trees more complex to increase accuracy in the autonomous decision making. The decision is based on the number of decision trees, data input, and the weight of each decision. This allocation of weight is linked to the medical database and based on the input from the user on their symptoms the software platform will finalize a treatment plan. The Random Forest algorithm, as implemented in the software platform of the present invention, could only be executed on a computing device such as that implemented in the present invention.

In some embodiments, the present invention is capable of analyzing test results, determining a treatment method based on an ailment diagnosed by the diagnostic module (400) and information from the medical records (2100), providing to the user a pharmacy capable of providing the prescription, pricing information for the prescription, and a plurality of recommendations, monitoring the user's health and recovery as the user carries out the treatment (475) determined by the diagnostic module (400), and issuing a request for the prescription determined by the treatment module (700) to the pharmacy fully autonomously.

The Diagnostic Module (400) may flag the User (100) with a high probability of being a diabetic based on running the symptoms by the diagnostic tables. To verify and classify what type of diabetes and how bad the patient's condition is, the system may request from the User (100) to use Glucometer from the patient's kit to measure the sugar level randomly. In addition, the system may ask the patient to perform a fasting blood sugar test using the Glucometer. The fasting blood sugar test may be an oral glucose tolerance test in the lab if the patient is a pregnant woman for example. Those tests may be determined based on the test and procedures table. The results of the tests and procedures may be read and assessed by the Diagnostic Module (400). Test evaluation table under the Test Analyzer Module may provide interpretation to the result of the medical test.

The Diagnostic Module (400) may have the capability to conduct visual diagnosis. For example, if the patient complains about a red spot. The system may ask the patient to take a picture using a high-resolution camera and the system may use Machine Learning to determine the possible condition. If the system couldn't provide a diagnosis with more than 99% probability. The system may request the patient to see a physician and the system may assist in identifying the appropriate physician and making appointments with him.

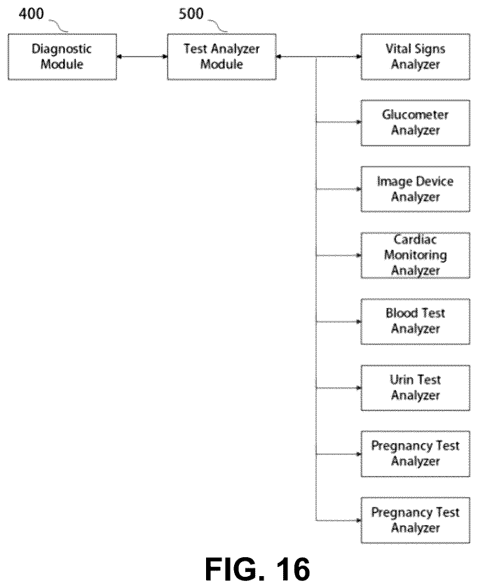

FIG. 16 shows the Test Analyzer Module, where the Diagnostic Module (400) may determine the required test and the Test Analyzer Module (500) may conduct the analysis based on predefined criteria. The Test Analyzer Module (500) may read the input from various peripherals including Personal Vital Signs, Personal EKG, Glucometer, Ultrasound Image Device, Cardiac Monitoring, and Skin/Eye Image Camera and additional handheld medical devices. The readings from those devices could be via Bluetooth, Zigbee, WiFi, and NFC. It also reads the external tests including lab test results and other forms of tests such as MIR images either through email, direct link or entered by the patient either verbally or in writing. The analysis of the test results shall take place in this module, where all the information from different medical equipment is converted into the same format and fed to the Diagnostic Module (400). The results of the analysis could be fed to the Medical Record Module (2100) and the Central Research Module (1200). The Test Analyzer Module (500) has multiple functionalities and each function could conduct certain analyses. The following are some functionalities that may be fed to the Test Analyzer Module (500):
i. Vital Signs Analyzer: This function processes the readings of the vital signs and correlates the vital signs readings with other variables to assess the patient condition and feed the results to the Diagnostic Module (400). The readings of the vital signs may be communicated to the Medical Record Module to log the input with the date and the time.
ii. Glucometer Analyzer: this function processes the readings of the blood surge and feeds the results to the Diagnostic Module (400). The various readings of the blood surge are recorded with time where analysis could be conducted. Accordingly, the Diagnostic Module (400) could adjust the treatment plan or the doses of insulin for example.
iii. Image Device Analyzer: this function may employ Machine Learning and image recognition to determine the results of reading for various types of images including ultrasound images, skin images, x-ray images, and eye images.
iv. Cardiac Monitoring Analyzer: this function may use an algorithm to perform the necessary analysis for cardiac patterns. With more usage from the user (100), more information is gathered and any abnormalities can be detected at an earlier stage.
v. Blood Test Analyzer: this function may determine the normal range and relevant analysis based on the results of various blood tests.
vi. Unrein Test Analyzer: this function may determine any abnormality in the tests and analyze the various unrein tests.
vii. Pregnancy Test Analyzer: this function may monitor the menstrual period of a female user (100), to determine the ovulation date for the highest chance of pregnancy and to detect early pregnancy to notify the female user (100).

In addition to the above, in general, all tests relevant to cellular and chemicals tests, diagnostic imaging, genetic testing, physical and visual examination and measurements (e.g. lumbar puncture) could be analyzed under Test Analyzer Module 500. Additional functionalities could be added as technology improves and reduces the size of analyzers to be more portable.

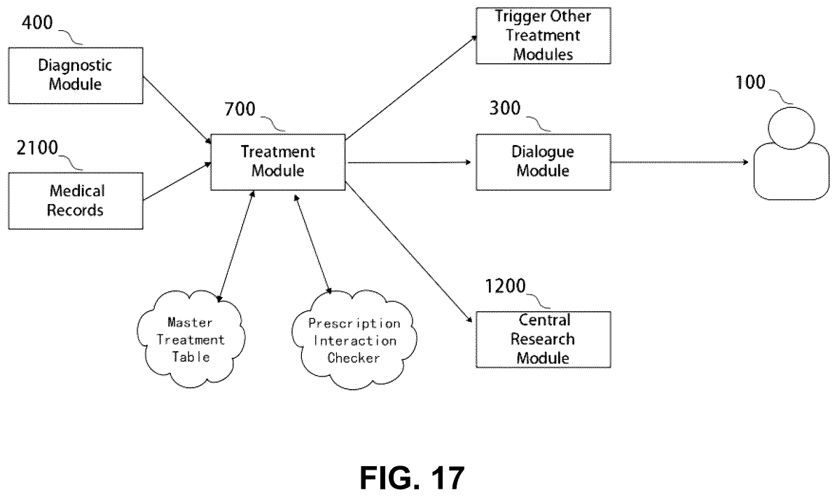

FIG. 17 shows the Treatment Module (700). The Treatment Module (700) takes the input from the Diagnostic Module (400) and Medical Records (2100) to determine the suitable treatment and prescriptions for the user (100). For example, the Medical Records (2100) could show that the user (100) has an allergy for certain prescriptions. The Treatment Module 700 in such cases could find an alternative medicine. Once the medical condition is determined by the Diagnostic Module (400) with high probability, the Treatment Module (700) may determine the appropriate treatment. For each condition (disease), there may be a predefined treatment plan and recommendation in a repository.

The treatment plan for each condition should be adjusted to suit the patient and to consider all relevant factors related to the patient such as age, medical history, and other diseases. The dose of the medicine may also be calculated based on those factors. For example, if the Diagnostic Module (400) performed tests and determined the user (100) condition is type 2 diabetes, the Treatment Module (700) may provide the patient with the proper treatment plan. The treatment repository may provide how to manage type 2 diabetes with the following factors to be considered:
Weight Control
Healthy eating
Regular exercise
Possibly, diabetes medication or insulin therapy
Blood sugar monitoring

The above treatment/recommendations may reside in the repository under the treatment of type 2 diabetes table. For each of the above variables, a table with more details may be available. For example, weight control and healthy eating as treatment recommendations are common in many diseases. Therefore, weight control and healthy eating may be treated as a standalone variable. At the same time, weight control should be defined and similarly healthy eating. Therefore, for weight control as a variable, there may be a table to determine what is the perfect weight based on relevant factors such as height, gender, body mass index (BMI), and age.

For healthy eating, the Treatment Module (700) may trigger the Dietitian Module (1100), and similarly for additional exercise and physical therapy the Treatment Module (700) may instruct the user (100) to the Physical Activity Module (1400). The Treatment Module (700) may instruct the user (100) with the frequency of checking the blood sugar and the system may monitor the blood sugar and maintain the readings. The Treatment Module (700) may also prescribe the suitable medication based on the user (100) condition. Other recommendations based on the user (100) condition may also be extended. For example, if the patient's body mass index (BMI) is greater than 35, the Treatment Module (700) may recommend weight-loss surgery (bariatric surgery).

The Treatment Module (700) may not just provide the recommendation but may develop a treatment plan suitable to the patient. For example, if the user (100) weight is average, the Treatment Module (700) may not have it as part of the treatment plan. However, the Treatment Module (700) may direct the User (100) to the Dietitian Module (1100) to assess whether the user (100) may follow healthy eating habits and may provide examples and recommendations of what can improve the healthy eating habits. The Master Treatment table is the repository for all treatments. For each condition (illness), there is a treatment plan which includes general recommendations, options, medicine, and alternative medicine. The treatment plan for each condition may be adjusted to suit the user (100).

The general recommendations may be provided to the patient through Dialogue Module (300) as a conversation. For each variable, more details may be retained to provide the patient with details. The medicine may be generic in the Master Treatment Table. Also, there could be many medicines for the same condition (illness). The Treatment Module (700) should choose the most suitable medicine based on factors relevant to the user (100). When there are multiple medicines the primary medicine may be flagged and prescribed. For example, if the condition is hypertension Thiazide Diuretics may be prescribed since it has fewer side effects. The Treatment Module (700) can trigger the Case Monitoring Module (800) for some conditions to determine the effectiveness of the prescription. In case the prescription is not effective the Case Monitoring Module (800) may request the Treatment Module (700) for alternative solutions.

There may be a table of brands of medicine. For each brand, there may be a dosage calculator. The calculator may determine the dosage including the frequency and duration of using the medicine based on relevant factors such as age, weight, and severity of the condition. The age and the weight of the patient may be imported from the User Account Module (200) and the severity of the condition may be imported from the Diagnostic Module (400). The side effect and direction of the prescription may be available to be shared with the user (100) before taking the prescription.

The Treatment Module (700) may initialize a drug interaction checker to check the user (100) other prescriptions, medical records (2100), or special conditions such as pregnancy to the conflicting medicine field to flag the conflicting prescription or a condition that could prevent taking the prescription. If the conflicting prescription is flagged, the Module may search for alternatives either for the condition under treatment or other prescriptions. The price of the prescription may also be available and the Treatment Module (700) may always suggest the cheapest brand as the first option and account for the user (100) insurance coverage when the side effects and the other factors of the various brands are the same.

The availability field may determine where the medicine can be collected. The Treatment Module (700) may give the patient the option to order the prescription directly from the pharmacy to be delivered or to be picked up. The availability field may provide all pharmacies within the vicinity of the patient where the prescription is available. The request of the prescription to the pharmacy may be processed through the External Communication Module (900). The rating field may collect the effectiveness of the prescription based on other patients'input and the results collected from the Case Monitoring Module (800). The rating filed could help in conducting research and it may be linked to the Central Research Module (1200).

FIG. 17 illustrates the Case Monitoring Module (800). Once a User (100) is diagnosed and begins the treatment. The software platform (600) may monitor the user (100) recovery and health through this module. Each treatment has a treatment cycle before the user (100) is fully recovered. At the same time, the user (100) could encounter some complications or the prescribed treatment could be ineffective. The software platform (600) may monitor the user (100) throughout the recovery process and for each treatment, the expected progress of the treatment may be tabulated in the Medical Records (2100).

For example, if the diagnosis of the condition was an infection and the software platform (600) made a prescription for certain antibiotics, the Case Monitoring Module (800) may compare the expected recovery time to the actual recovery time. If the recovery did not occur as expected the Case Monitoring Module (800) may provide a secondary course of action, which could include changing the antibiotics or conducting more tests with the Diagnostic Module (400) and the Test Analyzer Module (500).

The input for the Case Monitoring Module (800) may be based on the user (100) inputs and the results from the Test Analyzer Module (500). The Case Monitoring Module (800) could even request additional diagnostic measures through the Diagnostic Module (400). The Case Monitoring Module (800) may continue monitoring the condition until the user 100 is fully recovered. In some cases, the condition could be chronic such as hypertension and the Case Monitoring Module (800) may monitor the condition and provide the user (100) with recommendations and make adjustments to the prescription. The data collected from all users (100) of the Case Monitoring Module (800) could provide a significant improvement in the medical field and the data collected may be stored in the Central Research Module (1200).

The Case Monitoring Module (800) may import the prescription information prescribed by the Treatment Module (700) and may provide a reminder to the user (100) on when to take the prescription. Also, the user (100) may be reminded to refill the prescription before expiration or exhaustion of the prescription. The reminder could be in various forms including verbal messages, audible alerts, or text messages. The Case Monitoring Module (800) may have a Master Table of all the treatments and the expected progress of the results to assess the effectiveness of the treatment. For example, if the Diagnostic Module (400) determined the condition as hypertension and the Treatment Module (700) prescribed Thiazide diuretics as the medicine for treatment and if the condition based on the readings of the pressure over one week after taking Thiazide diuretics continues not to be stable. The Case Monitoring Module (800) may trigger the Treatment Module (700) to find another alternative treatment.

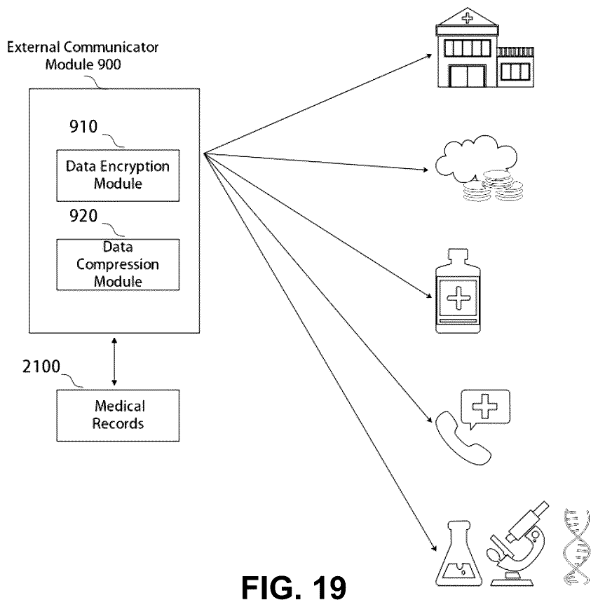

FIG. 19 shows the External Communicator Module (900). The External Communicator Module (900) links the software platform (600) with all external parties including clinics, hospitals, emergency centers (ambulances), and pharmacies. The External Communicator Module (900) also consists of a Data Encryption Module (910) and Data Compression Module (920). The Data Encryption Module (910) translates all data in different formats to a standardized usable format when receiving information from hospitals, labs, clinics, emergency centers, and pharmacies. At the same time, the Data Encryption Module (910) converts outgoing data to a format that is readable and usable for hospitals, labs, clinics, emergency centers, and pharmacies. Before any data is transferred out, the Data Encryption Module (910) encrypts all data collected from the User (100) in accordance with industry standards, or blockchain and then compresses the encrypted data utilizing the Data Compression Module (920) in accordance with medical industry-standard compression, like Diacom.

The Data Encryption Module (910) may exchangeably be referred to as a Data Conversion Module. In some embodiments, the Data Encryption Module (910) may be capable of determining the data source format (e.g. PDF, Excel, etc.) and convert the data to a different file format. Data Encryption Module (910) may be an artificial intelligence software that is used for data mining by taking different medical formats and gathering the information to convert it into a usable single database. The various formats can be encrypted, which will require decryption. The data can be in different locations within the file and without the data conversion module, a person doing this task would need to spend a lot of time and effort to go through the various formats for each user.

For the solution to work effectively, external parties including hospitals, pharmacies, labs, and emergency centers, and nursing service providers should register with the system to receive requests and share data. Different tasks should be able to be executed and data to be shared by each party. Pharmacies should be able to share pricing information including sale items with the External Communicator Module (900) and the Payment Gateway (2400) may be able to communicate with each of the external parties to make payment when payments are due. Hospitals scheduling systems should be compatible with the solution so the appointment could be booked automatically or in a format that can be translated by the Data Encryption Module (910). The medical records (2100) of the hospital should also be able to feed the system and vice versa.

Pharmacies should be able to receive prescriptions developed by the Treatment Module (700) with instructions such as a location for the delivery of the prescription. Labs scheduling systems should also be connected to the solution so an appointment can be scheduled. The test and procedures determined by the Diagnostic Module (400) should be delivered to the lab. At the same time, the lab either uploads the results or the Lab system could be accessible by the Test Analyzer Module (500). Emergency Centers should be able to receive a request for an ambulance with a description of the emergency and the user (100) location should be sent automatically from the smartphone or the standalone device. In a simplistic format and before the adoption of the solution by the various medical facilities, the system should be able to conduct an auto call to the emergency center (e.g. 911) and request help through voice message with the location. Such emergency calls should be triggered either by the user (100) or as a result of vital signs readings when the user (100) is susceptible to emergency for example heart attack.

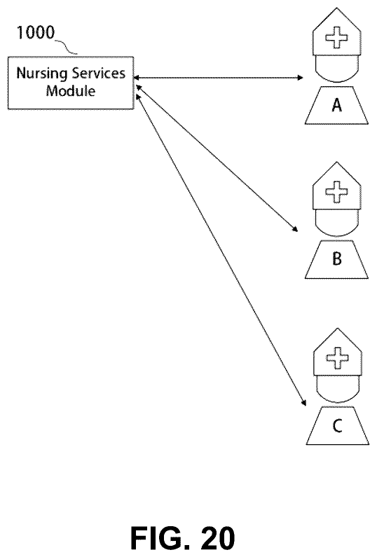

FIG. 20 shows the Nursing Services Module (1000). The Nursing Services Module (1000) may work with registered nurses or licensed health care providers to sign in and be available to be hired by the user (100) as an on-demand service. Volunteers could also register to render their services to the user (100) in need and receive a log of service hours provided, where the volunteer can declare this as charity work, pro bono, or community service. The user 100 could look up available nurses or volunteers in his/her neighborhood or could initiate an ad with his/her needs and the nurse or volunteer who is interested could respond and apply to offer their services.

Those who are interested to offer their medical services, whether they are volunteers or registered nurses should register via an application or through a web page. Basic information should be entered with a brief biography as well as their photo, licenses, and resume. The registered nurse or volunteer may go through a background check. The background check could take various scenarios such as a recommendation from an institution or security office. The background check is important for the security of the user (100) and to ensure the minimum necessary experience as a service provider. Once the registration is completed, the nurse or the volunteer could trigger his or her availability along with his or her location of coverage. When the user (100) requests nursing services, the application may display all available service providers in the neighborhood. The nurses could determine their hourly rates while volunteers could provide the number of hours that they are willing to allocate. Upon selection of the nurse by the user (100), a message may be sent to the nurse with brief information about the patient's condition, history, the patient location, and the sought services. The nurse could decline or accept the assignment. Then the nurse or service provider may be dispatched to the user (100) location and after the service, the user (100) can rate the service provider where the rating may be shared with other users.

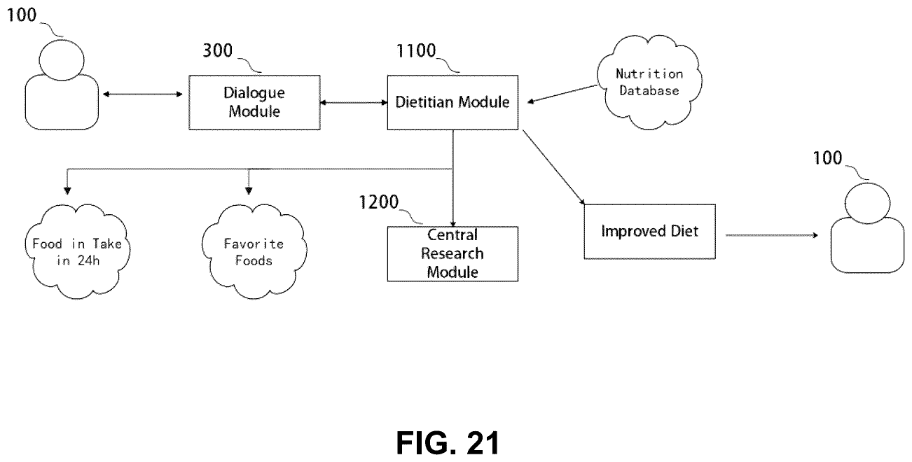

FIG. 21 shows the Dietitian Module (1100). The Dietitian Module (1100) may play the role of the dietitian and may put a plan that fits health goals, food preferences, and lifestyle. For example, the Dietitian Module (1100) could monitor user (100) carbohydrate intake and determine the amount of carbohydrates they need to intake with each meal and snack to keep their blood sugar levels more stable.

The Dietitian Module (1100) may conduct an initial assessment of the user (100) eating habits along with a check on the Medical Records (2100). The dialogue that may take place between the user (100) and the Dietitian Module (1100) is to complete an eating habits assessment form. A questionnaire may be filled based on the input of the user (100). The purpose of the questionnaire is to determine the food that the patient eats, the quantities, and preferences. For example, the Dietitian Module (1100) may ask the user (100) the following questions:
How many meals do you have every day?
Usually what do you have in each meal?
What did you have yesterday?

For some responses, the Dietitian Module (1100) may request the quantities if the quantities are not given. Also, the quantities could be in a descriptive form. For example, if the user (100) mentioned that he eats cashew nuts, the Dietitian Module (1100) may help the user (100) to provide a rough estimate of the quantities by accepting answers like a handful, or a few bites. The response of the user (100) may be entered into two tables. One is the user (100) food of the last 24 hours (an example is displayed in Table 6) and the typical food that the user 100 usually eats. Many individuals have non-routine food they eat. The Dietitian Module (1100) may extract such information and tabulate them for analysis.

The Dietitian Module (1100) may have a table of the ingredients and calories of all foods. Based on the user (100) input the Dietitian Module (1100) may convert the food into calories and ingredients. For example, if the patient eats cashew nuts regularly and if the average of the user (100) intake is around 100 mg the Dietitian Module (1100) may convert the 100 gm of cashews to its ingredient. When the user (100) enters a recipe such as pasta, the system may have a table with the breakdown of the recipe. If the dish is not predefined, the Module may ask the user (100) to provide the ingredients of the dish. Based on the analysis of the ingredient intake the Dietitian Module (1100) may identify the elements such as a certain vitamin that the patient does not take sufficient quantities and may make recommendations of foods that contain the missing elements.

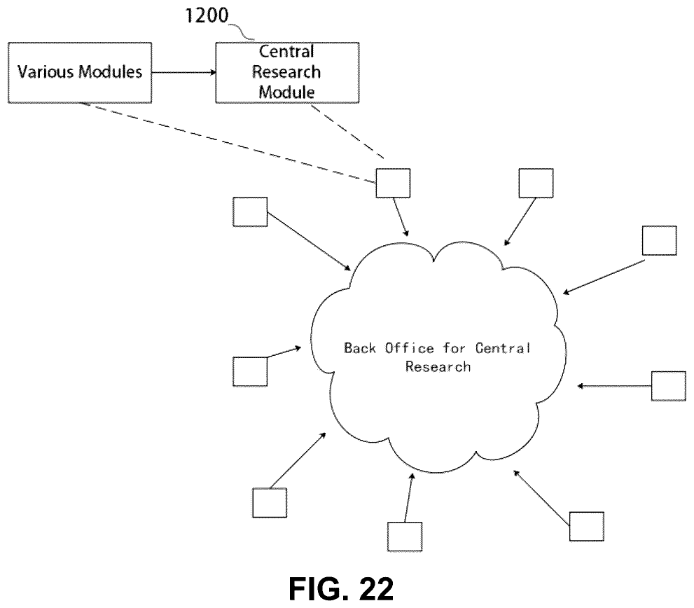

FIG. 22 shows the Central Research Module (1200). The Central Research Module (1200) operates in the back office, where data is collected for medical research. Mass data collected from all the software platform (600) users, the effectiveness of medicines from the Case Monitoring Module (800) could be improved. By autonomously analyzing the correlation between the tests and the diagnoses for the user (100) the unnecessary tests could be eliminated. the illness and treatment of each user (100) are analyzed. The collected data may be used to segregate users (100) by ethnicity and DNA to analyze the probability of recurring illnesses and by utilizing data science along with Machine Learning (M.L.) to provide preventive medicine. The data could be available as an open-source for researchers globally.

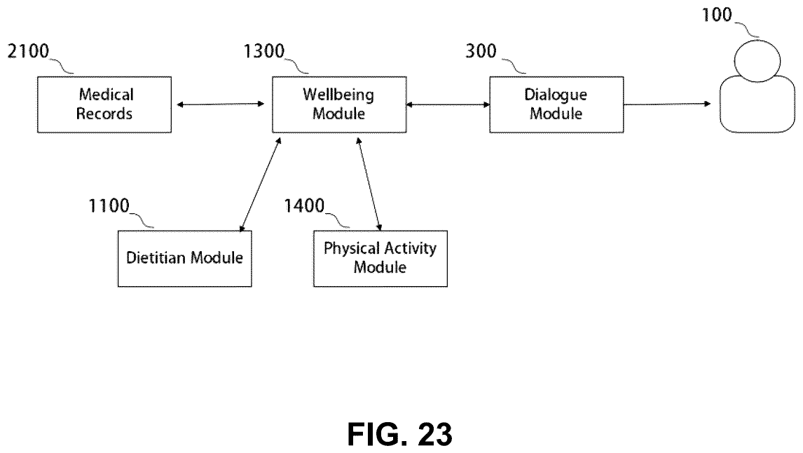

FIG. 23 shows the Wellbeing Module (1300). The Wellbeing Module (1300) may provide general recommendations that could improve user (100) wellbeing, working in cooperation with the Dietitian Module (1100) and the Physical Activity Module (1400) to recommend certain supplements that are determined based on the user (100) special needs taking into consideration of age, physical condition and the Medical Records (2100) of the user (100) including any genetically inherited diseases.

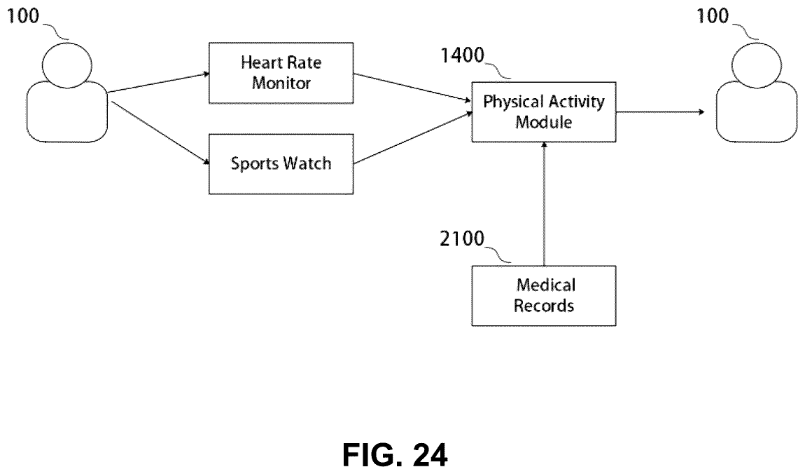

FIG. 24 shows the Physical Activity Module (1400). The Physical Activity Module 1400 may provide physical activity tracking and instructions to the user (100). The Physical Activity Module (1400) can track and collect data on the amount of exercise the user (100) performed in a day and provide feedback on the result to instruct the user (100) to make improvements to obtain the result the user (100) is seeking.

The Physical Activity Module (1400) can also assist the user (100) with physical therapy and improve the user (100) range of motion through instructional videos and monitoring the user (100) vital signs.

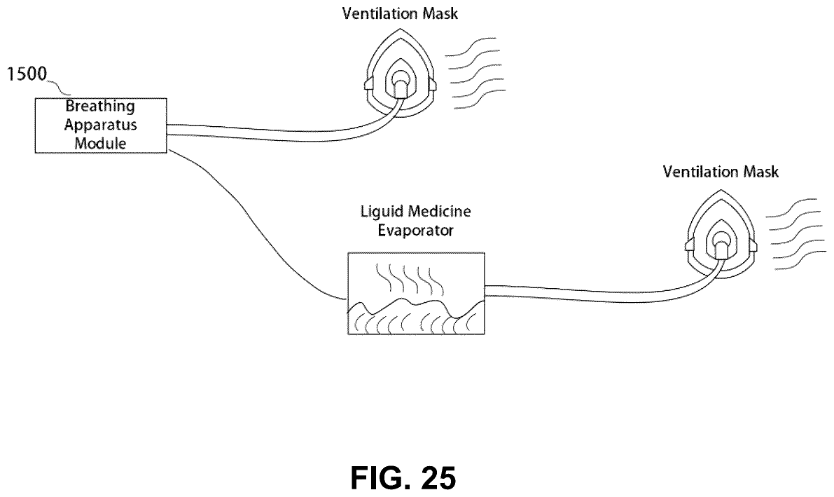

FIG. 25 shows the Breathing Apparatus Module (1500). The Breathing Apparatus Module (1500) can function as a ventilator that moves breathable air into a non-invasive ventilation mask or bag valve mask to deliver breaths to the user (100) who is physically unable to breathe or is breathing insufficiently. The Breathing Apparatus Module (1500) can also function as a nebulizer to assist users (100) that have asthma by delivering liquid medicine via mist through the ventilation mask. The Breathing Apparatus Module (1500) can be set up initially as a long-term breathing support system for the user (100) from the beginning or can be activated by the user (100) if the user (100) requests symptoms that require breathing support.

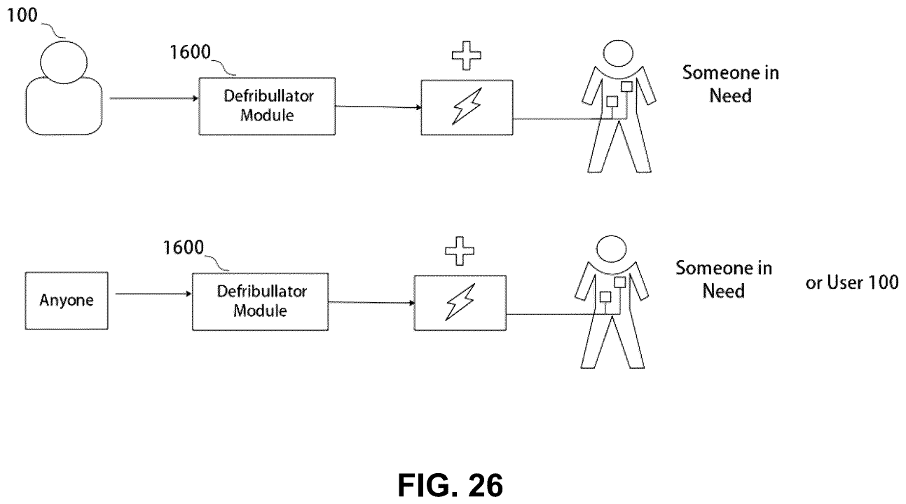

FIG. 26 shows the Defibrillator Module (1600). The Defibrillator Module (1600) is a treatment for life-threatening cardiac dysrhythmias by delivering a dose of electric current to the heart. This module can be used as an emergency device by any user to help someone in need. Or can be utilized by someone to assist the user (100) who might require emergency medical assistance. In an emergency, the software platform (600) can instruct the User (100) or a person who can provide the emergency care through the Dialogue Module (300) to utilize the Defibrillator Module (1600). Turning the software platform (600) as an emergency device to save other people. When the platform based on the readings of the vital signs determines the condition of the patient as “code blue” the software platform (600) may activate a siren in addition to activating a call to the emergency center, and the software platform (600) may instruct the User (100) or a person who can provide the emergency care on how to use the defibrillator to perform emergency medical service to the person in need or the user (100).

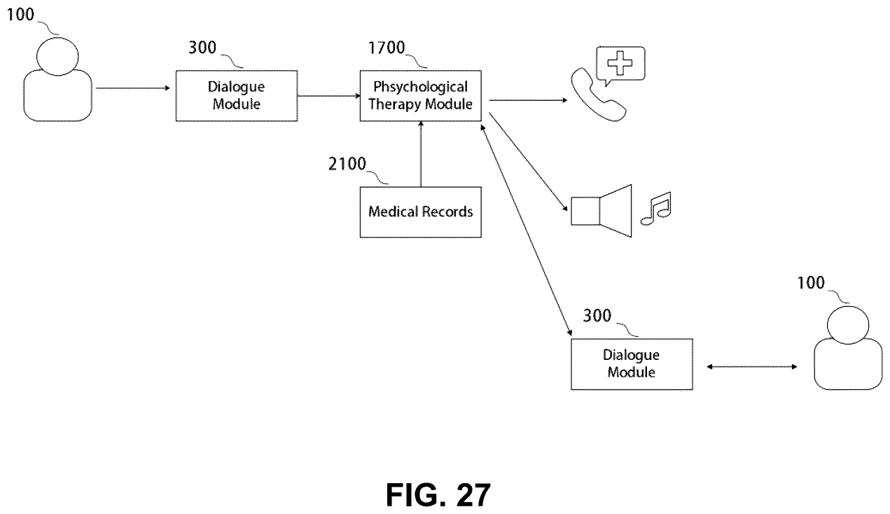

FIG. 27 shows the Psychological Therapy Module (1700). The Psychological Therapy Module (1700) may provide a diagnostic and psychological assessment to User (100) to determine the condition and extend recommendation. The facial sentiment and voice tone could be used to assess the progress of the condition. Utilizing the Dialogue Module (300) the tone of the User (100) voice is analyzed to determine if the User (100) is in distress, pain, anxiety, depression, or anger. The software platform (600) may utilize machine learning to adapt to the User (100), with a base built from the User Account Module (200) to determine the User (100) medical records (2100), other reported habits and conditions. The User (100) can utilize the Dialogue Module (300) to tell the software platform (600) their emotional and psychological issues. For example, the User (100) can be trembling with a shaky voice, shortness of breath that is impacting their speech, sweating, and complaining of chest pain and choking. The Diagnostic Module (400) may immediately conclude this is an anxiety or panic attack and respond to the User (100) to perform self-therapy of breathing exercises, at the same time provide positive encouragement with a soothing voice and music or natural sounds. In a more severe situation, a User (100) can speak to the software platform (600) that they want to commit suicide. The software platform (600) may immediately notify emergency services and distract the User (100) with questions, positive encouragement to give emergency services time to reach the User (100).

The platform may analyze the physical condition which in many cases could affect the Psychology of the person. The Diagnostic Module may be employed to trace the possibility of physical conditions such as an imbalance of chemicals that could affect psychology. A similar diagnostic table to the physical diagnostic table may be triggered when the patient complaint is relevant to sentimental concerns. After the diagnostic, and conducting certain predefined physiological tests the system may trigger the Treatment Sub-Module where various treatment plans may be determined. In another instance, the User (100) can have long-term psychological therapy utilizing the Psychological Therapy Module (1700). The therapy could be self-improvement, building confidence, overcoming fear, listening to an audiobook, or simply speaking to the software platform (600) to offload stress and peace of mind. The software platform (600) may collect this User (100) data and through machine learning track the User (100) improvement and adjust therapy sessions.

The present invention features a cloud connected diagnostic module (400) comprising a secure communication interface for retrieving user specific information, disease profiles, symptoms data, decision trees, and treatment information from a remote server and logic for generating probability mappings of symptoms to potential medical conditions to treatments using the retrieved disease data and the user specific information. The present invention features a portable autonomous medical evaluation platform implemented on a hardware device and configured to receive sensor inputs locally or wirelessly, apply on-board diagnostic logic corresponding to a diagnostic module (400), and generate treatment recommendations based on the sensor inputs without cloud dependency.

The present invention features a method for autonomous healthcare comprising. The method may comprise receiving symptoms and external medical records in standardized format using an external communicator module (900). The method may further comprise analyzing input data using a diagnostic module (400). The method may further comprise generating a treatment plan using a treatment module (700). The method may further comprise issuing electronic prescriptions or test requests. The method may further comprise adjusting treatment using a case monitoring module (800) based on monitored recovery data. The method may further comprise ordering prescriptions and scheduling lab tests automatically. The method may further comprise monitoring recovery through periodic data collection. The method may further comprise adjusting diagnosis and treatment protocols in real-time based on collected data. The method may further comprise initiating emergency services when critical health parameters are detected. The method may further comprise integrating external lab results and images into the diagnostic module (400).

The present invention features a means for containing and executing a virtual physician software comprising a non-transitory computer-readable medium for containing and executing an autonomous virtual physician autonomous software platform (600) for integrating various services utilizing artificial intelligence and natural language. The non-transitory computer-readable medium may comprise hardware for storing and executing process algorithms, as well as embedded hardware components for sensing and communication capabilities.

The non-transitory computer-readable medium may comprise data comprising a user account module (200) comprising instructions for storing general information (2000) of a user in a hardware memory component, receiving medical records (2100) of the user from an external communicator module (900) through a hardware communication component (e.g., an antenna, a coaxial cable, any component that allows for an Internet connection), storing the medical records (2100) of the user in a hardware memory component, storing reported conditions (2200) of the user in a hardware memory component, storing user habits (2300) of the user in a hardware memory component, and transmitting a payment to an external source through the use of a payment gateway (2400) connected to by the hardware communication component.

The non-transitory computer-readable medium may further comprise data comprising a dialogue module (300) comprising instructions for communicating verbally with the user through the use of a verbal input hardware component and Natural Language Processing algorithms contained as data in the non-transitory computer-readable medium, and determining a software module to transfer action based on directions from the user received through an input hardware component.

The non-transitory computer-readable medium may further comprise data comprising a diagnostic module (400) comprising instructions for accessing a disease database of disease symptoms mapped to possible causes stored either in physical memory or in a cloud computing server connected to through the hardware communication component, accepting a list of user symptoms provided by the user to the dialogue module (200), accepting a set of test results from a test analyzer module (500), generating a first table (410) mapping each of the user symptoms to all possible causes for the user symptom, generating a second table displaying all possible causes (450) that share every one of the user symptoms, and accepting data from a database of medical records (2100). The database of medical records (2100) may comprise medical records connected to the user received from a plurality of external medical sources (930) through the integrated hardware communication component, such that each medical record is converted to a standard format by the software upon being received by the virtual physician software. The instructions may further comprise generating a probability table ranking all possible causes (450) based on probability of the presence of each possible cause.

The non-transitory computer-readable medium may further comprise data comprising the test analyzer module (500) comprising instructions for accepting the set of test results from testing equipment, which are hardware components integrated into or communicatively coupled to the non-transitory computer-readable medium for sensing capabilities, autonomously analyzing the set of test results, transmitting the analyzed set of test results to the user account module (200) to be stored, and transmitting the analyzed set of test results to the diagnostic module (400).

The non-transitory computer-readable medium may further comprise data comprising a treatment module (700) comprising instructions for receiving input from the medical records (2100) stored in the user account module (200) and the diagnostic module (400), and autonomously determining a treatment method based on an ailment diagnosed by the diagnostic module (400) and information from the medical records (2100). The treatment method may comprise a prescription and a plurality of recommendations, and autonomously providing to the user a pharmacy capable of providing the prescription, pricing information for the prescription, and the plurality of recommendations.

The non-transitory computer-readable medium may further comprise data comprising a case monitoring module (800) comprising instructions for autonomously monitoring the user's health and recovery as the user carries out the treatment (475) determined by the diagnostic module (400). This can be done through accepting input directly from the user or supervisor, or sensor input from a sensing device used by the user and communicatively coupled to the non-transitory computer-readable medium. The instructions may further comprise reminding the user to carry out the prescription related to the treatment (475) through visual or audio output provided through an auxiliary component communicatively coupled to the non-transitory computer-readable medium (e.g., a smartphone, speakers, a screen), comparing an expected recovery time to an actual recovery time to determine if a secondary course of action is necessary, adjusting the prescription as the secondary course of action, and adjusting the expected recovery time based on the secondary course of action.

The non-transitory computer-readable medium may further comprise data comprising the external communicator module (900) comprising a data encryption module (910) comprising instructions for accepting data from a source through a hardware communication component, and computationally encrypting the data into a standard format unreadable by a human user. The data encryption module (910) may be capable of accepting data directly from the user, or from the plurality of external medical sources (930). The external communicator module (900) may comprise a data compression module (920) comprising instructions for accepting standard format data from the data encryption module (910), compressing the standard format data into compressed data, and transmitting the compressed data to an external storage (9000) component or to the plurality of external medical sources (930). The external communicator module (900) may comprise instructions for autonomously issuing a request for the prescription determined by the treatment module (700) to the pharmacy provided by the treatment module (700) through the use of a hardware communication component.

The non-transitory computer-readable medium may further comprise data comprising a dietician module (1100) comprising instructions for allowing the user to input food consumption data through the use of a hardware input component, generating a table of calorie intake based on the food consumption data, and transmitting the food consumption data to the user account module (200). The non-transitory computer-readable medium may further comprise data comprising a wellbeing module (1300) comprising instructions for providing recommendations for general health of the user based on information from the user account module (200) through the use of the one or more auxiliary components described above. The non-transitory computer-readable medium may further comprise data comprising a physical activity module (1400) comprising instructions for tracking physical activity of the user through one or more of the input components and methods described above, and providing recommendations for physical activity of the user based on information from the user account module (200), such that the recommendations are provided through one of the auxiliary components described above.

The present invention may further comprise a breathing apparatus module (1500) comprising a ventilation hardware device for aiding in the user's breathing, and a nebulizer hardware device for delivering medicine to a set of lungs of the user. These are physical components that can be used conjunction with the software components and modules described above. The present invention may further comprise a defibrillator module (1600) comprising a defibrillation device for electrically stimulating a heart of the user. The defibrillator module (1600) is a physical hardware component in communication with the non-transitory computer-readable medium that also acts as a non-transitory computer-readable medium comprising instructions for providing instructions on how to use the defibrillation device through a visual and/or audio component, and contacting, by the external communicator module (900), emergency medical aid upon detecting vital signs requiring immediate attention. The non-transitory computer-readable medium may further comprise data comprising a psychological therapy module (1700) comprising instructions for analyzing a psychological state of the user based on a tone of voice or facial expression of the user collected by the dialogue module (300), gathered by a input hardware component (e.g., a camera, a microphone, etc.), contacting, by the external communicator module (900), emergency medical aid upon the user expressing a desire to commit suicide, and providing long-term psychological therapy to the user by prompting repeated use of this module through one or more of the auxiliary components described above. Each module of the software may be capable of transmitting data to all other modules of the software. The non-transitory computer-readable medium may further comprise the disease database comprising disease symptoms mapped to possible diseases, a research database comprising disease data and recovery data from a plurality of users, and the external storage (9000) component.

The computer system can include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R. F, Bluetooth, storage media, computer buses, etc., or any suitable combination of the foregoing. Computer program code for carrying out operations for aspects of the present disclosure may be written in any combination of one or more programming languages, including an object oriented programming language such as Java, Smalltalk, C#, Ruby, or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft® .NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation®; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

The present invention may comprise or implement a neural network for machine learning tasks. The neural network may be stored, trained, and/or executed entirely on the user's computer, partly on the user's computer, as a stand-alone software package, partly on the user's computer and partly on a remote computer or entirely on the remote computer or server. The neural network may be stored in the form of program code, as described above. The neural network, in some embodiments, may be a perceptron neural network, a feed forward neural network, a multilayer perceptron neural network, a convolutional neural network, a radial basis functional neural network, a recurrent neural network, a long short-term memory neural network, a sequence-to-sequence neural network model, a modular neural network, or the like.

Although there has been shown and described the preferred embodiment of the present invention, it may be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. An autonomous virtual physician autonomous software platform (600) for integrating various services under one autonomous platform utilizing artificial intelligence and natural language, the platform comprising:
a. a means for containing and executing a virtual physician software, the virtual physician software comprising:
i. a user account module (200) comprising instructions for:
A. storing general information (2000) of a user,
B. receiving medical records (2100) of the user from an external communicator module (900),
C. storing the medical records (2100) of the user,
D. storing reported conditions (2200) of the user,
E. storing user habits (2300) of the user, and
F. transmitting a payment to an external source through the use of a payment gateway (2400);
ii. a dialogue module (300) comprising instructions for:
A. communicating verbally with the user through the use of a verbal input component and Natural Language Processing algorithms, and
B. determining a module to transfer action based on directions from the user;
iii. a diagnostic module (400) comprising instructions for:
A. accessing a disease database of disease symptoms mapped to possible causes,
B. accepting a list of user symptoms provided by the user to the dialogue module (200),
C. accepting a set of test results from a test analyzer module (500);
D. generating a first table (410) mapping each of the user symptoms to all possible causes for the user symptom,
E. generating a second table displaying all possible causes (450) that share every one of the user symptoms,
F. accepting data from a database of medical records (2100), wherein the database of medical records (2100) comprises medical records connected to the user received from a plurality of external medical sources (930), such that each medical record is converted to a standard format upon being received by the virtual physician software, and
G. generating a probability table ranking all possible causes (450) based on probability of the presence of each possible cause;
iv. the test analyzer module (500) comprising instructions for:
A. accepting the set of test results from testing equipment,
B. autonomously analyzing the set of test results,
C. transmitting the analyzed set of test results to the user account module (200) to be stored, and
D. transmitting the analyzed set of test results to the diagnostic module (400);
v. a treatment module (700) comprising instructions for:
A. receiving input from the medical records (2100) stored in the user account module (200) and the diagnostic module (400),
B. autonomously determining a treatment method based on an ailment diagnosed by the diagnostic module (400) and information from the medical records (2100), wherein the treatment method comprises a prescription and a plurality of recommendations, and
C. autonomously providing to the user a pharmacy capable of providing the prescription, pricing information for the prescription, and the plurality of recommendations;
vi. a case monitoring module (800) comprising instructions for:
A. autonomously monitoring the user's health and recovery as the user carries out the treatment (475) determined by the diagnostic module (400),
B. reminding the user to carry out the prescription related to the treatment (475),
C. comparing an expected recovery time to an actual recovery time to determine if a secondary course of action is necessary,
D. adjusting the prescription as the secondary course of action, and
E. adjusting the expected recovery time based on the secondary course of action;
vii. the external communicator module (900) comprising:
A. a data encryption module (910) comprising instructions for:
I. accepting data from a source, and
II. encrypting the data into a standard format,
wherein the data encryption module (910) is capable of accepting data directly from the user, or from the plurality of external medical sources (930);
B. a data compression module (920) comprising instructions for:
I. accepting standard format data from the data encryption module (910),
II. compressing the standard format data into compressed data, and
III. transmitting the compressed data to an external storage (9000) component or to the plurality of external medical sources (930);
wherein the external communicator module (900) comprises instructions for:
C. autonomously issuing a request for the prescription determined by the treatment module (700) to the pharmacy provided by the treatment module (700);
viii. a dietician module (1100) comprising instructions for:
A. allowing the user to input food consumption data,
B. generating a table of calorie intake based on the food consumption data, and
C. transmitting the food consumption data to the user account module (200);
ix. a wellbeing module (1300) comprising instructions for:
A. providing recommendations for general health of the user based on information from the user account module (200);
x. a physical activity module (1400) comprising instructions for:
A. tracking physical activity of the user, and
B. providing recommendations for physical activity of the user based on information from the user account module (200);
xi. a breathing apparatus module (1500) comprising:
A. a ventilation device for aiding in the user's breathing, and
B. a nebulizer for delivering medicine to a set of lungs of the user;
xii. a defibrillator module (1600) comprising a defibrillation device for electrically stimulating a heart of the user and comprising instructions for:
A. providing instructions on how to use the defibrillation device, and
B. contacting, by the external communicator module (900), emergency medical aid upon detecting vital signs requiring immediate attention; and
xiii. a psychological therapy module (1700) comprising instructions for:
A. analyzing a psychological state of the user based on a tone of voice or facial expression of the user collected by the dialogue module (300),
B. contacting, by the external communicator module (900), emergency medical aid upon the user expressing a desire to commit suicide, and
C. providing long-term psychological therapy to the user;
wherein each module of the software is capable of transmitting data to all other modules of the software;
b. the disease database comprising disease symptoms mapped to possible diseases;
c. a research database comprising disease data and recovery data from a plurality of users;
d. the external storage (9000) component.

2. The autonomous virtual physician software platform (600) of claim 1, wherein the external communicator module (900) further communicates with:
a. a hospital to receive medical records of the user and scheduling information for arranging an appointment, and transmits the user data and appointment requests to the hospital;
b. a medical lab to receive medical records of the user, scheduling information for arranging an appointment, and lab results, and transmit the user data and appointment requests to the medical lab;
c. a clinic to receive medical records of the user and scheduling information for arranging an appointment, and transmit the user data and appointment requests to the clinic;
d. an emergency center to request an ambulance, and transmit the user data and a location of the user to the emergency center; and
e. the pharmacy to receive medical records of the user and pricing information for the prescriptions, and transmit the user data, a plurality of the prescriptions, and a delivery location for the prescriptions to the pharmacy.

3. An autonomous virtual physician autonomous software platform (600) comprising testing equipment for allowing the user to carry out the plurality of tests for diagnosing a medical issue of a user, the platform comprising:
a. a means for containing and executing a virtual physician software, the virtual physician software comprising:
i. a database of medical records (2100) comprising:
A. a medical history of the user,
B. a list of pre-existing conditions of the user, and
C. a list of medications currently taken by the user,
wherein data found in the database of medical records (2100) is received from a plurality of external medical sources (930), such that each medical record is converted to a standard format upon being received by the virtual physician software;
b. a diagnostic module (400) comprising instructions for:
i. accessing a disease database of disease symptoms mapped to possible causes,
ii. accepting a list of user symptoms from the user,
iii. generating a first table (410) mapping each user symptom to all possible causes for the user symptom,
iv. generating a second table displaying all possible causes (450) that share every user symptom,
v. accepting data from the database of medical records (2100) received from the plurality of external medical sources (930),
vi. generating a probability table ranking all possible causes (450) based on probability of the presence of each possible cause,
vii. requesting a plurality of tests from the user to better determine the possible cause,
viii. accepting a set of test results from the user,
ix. narrowing down the probability table based on the set of test results, and
x. autonomously determining a treatment (475) for the possible causes based on the data from the database of medical records (2100), the list of user symptoms, and the set of test results by executing a Random Forest Classifier algorithm;
wherein each module of the software is capable of transmitting data to all other modules of the software; and
c. the disease database comprising disease symptoms mapped to possible diseases.

4. The autonomous virtual physician software platform (600) of claim 3, wherein the testing equipment comprises a personal vital sign monitor, a personal EKG, a glucometer, an ultrasound imaging device, a cardiac monitor, and a skin image camera.

5. The autonomous virtual physician software platform (600) of claim 3 further comprising a test analyzer module (500) for accepting tests from the user through use of the testing equipment and processing the tests to generate the set of test results to aid the diagnosis platform in generating the possible cause.

6. The autonomous virtual physician software platform (600) of claim 3, wherein the database of disease symptoms is stored in a cloud server and accessed by the diagnostic module (400).

7. The autonomous virtual physician software platform (600) of claim 3, wherein the probability tables are generated for every user symptom.

8. The autonomous virtual physician software platform (600) of claim 3, wherein the probability weights are calculated by dividing the number of symptoms matched between the user symptoms and the symptoms of the possible cause by the total number of user symptoms and the symptoms are further narrowed down with tests and communications with the user to determine the most probable cause.

9. The autonomous virtual physician software platform (600) of claim 3, wherein the first table (410) is filtered using data from the database of medical records comprising an age, a gender, a weight, and a medical history of the user.

10. An autonomous virtual physician software platform (600) for compiling a user's medical history from a plurality of sources and using the medical history to diagnose a medical issue of a user, the platform comprising:
a. a means for containing and executing a virtual physician software, the virtual physician software comprising:
i. an external communicator module (900) for gathering information pertaining to the user's medical history from a plurality of sources, the external communicator module (900) comprising:
A. a data encryption module (910) comprising instructions for:
I. accepting data from a source, and
II. converting the data into a standard format, wherein the data encryption module (900) is capable of accepting data directly from the user, or from a plurality of external medical sources (930);
B. a data compression module (920) comprising instructions for:
I. accepting standard format data from the data encryption module (910),
II. compressing the standard format data into compressed data, and
III. transmitting the compressed data to an external storage component (9000) or to the plurality of external medical sources (930);
ii. a database of medical records (2100) received from the plurality of external medical sources (930) comprising:
A. a medical history of the user,
B. a list of pre-existing conditions of the user, and
C. a list of medications currently taken by the user,
wherein data found in the database of medical records (2100) is received from the plurality of external medical sources (930), such that each medical record is converted to a standard format upon being received by the virtual physician software; and
iii. a diagnostic module comprising instructions for:
A. accessing a disease database of disease symptoms mapped to possible causes,
B. accepting a list of user symptoms from the user,
C. generating a first table (410) mapping each user symptom to all possible causes for the user symptom,
D. generating a second table displaying all possible causes (450) that share every user symptom,
E. accepting data from the database of medical records (2100),
F. generating a probability table ranking all possible causes (450) based on probability of the presence of each possible cause,
G. requesting a plurality of tests from the user to better determine the possible cause,
H. accepting a set of test results from the user,
I. narrowing down the probability table based on the set of test results, and
J. autonomously determining a treatment (475) for the possible causes based on the data from the database of medical records (2100), the list of user symptoms, and the set of test results,
iv. a case monitoring module (800) comprising instructions for:
A. autonomously monitoring the user's health and recovery as the user carries out the treatment (475) determined by the diagnostic module (400),
B. reminding the user to carry out a prescription related to the treatment (475),
C. comparing an expected recovery time to an actual recovery time to determine if a secondary course of action is necessary,
D. adjusting the prescription as the secondary course of action, and
E. adjusting the expected recovery time based on the secondary course of action;
wherein each module of the software is capable of transmitting data to all other modules of the software; and
b. the disease database comprising disease symptoms mapped to possible diseases; and
c. the external storage (9000) component.

11. The autonomous virtual physician software platform (600) of claim 10, wherein the diagnostic module (400) further comprises instructions for requesting a plurality of tests from the user to better determine the possible cause.

12. The autonomous virtual physician software platform (600) of claim 11 further comprising testing equipment for allowing the user to carry out the plurality of tests.

13. The autonomous virtual physician software platform (600) of claim 12, wherein the testing equipment comprises a personal vital sign monitor, a personal EKG, a glucometer, an ultrasound imaging device, DNA sampler, a cardiac monitor, and a skin image camera.

14. The autonomous virtual physician software platform (600) of claim 11 further comprising a test analyzer module (500) for accepting tests from the user through use of the testing equipment and processing the tests to generate the set of test results to aid the diagnosis platform in generating the possible cause.

15. The autonomous virtual physician software platform (600) of claim 10 further comprising a treatment module (700) for autonomously generating a plurality of treatments for the plurality of possible causes generated by the diagnostic module (400) and allowing the user to select a route of treatment.

16. The autonomous virtual physician software platform (600) of claim 10, wherein the external communicator module (900) further comprises instructions for ordering the prescription to be delivered to the user.

17. The autonomous virtual physician software platform (600) of claim 10 further comprising a dietitian module (1100) for keeping track of the user's dietary habits as data to be added to the database of medical records (2100) and for aiding the user in maintaining a healthy diet, losing weight and to prevent medical interactions in accordance with the treatment determined by the platform.

18. A cloud connected diagnostic module (400) comprising a secure communication interface for retrieving user specific information, disease profiles, symptoms data, decision trees, and treatment information from a remote server and logic for generating probability mappings of symptoms to potential medical conditions to treatments using the retrieved disease data and the user specific information.

19. A portable autonomous medical evaluation platform implemented on a hardware device and configured to receive sensor inputs locally or wirelessly, apply on-board diagnostic logic corresponding to a diagnostic module (400), and generate treatment recommendations based on the sensor inputs without cloud dependency.

20. A method for autonomous healthcare comprising:
a. receiving symptoms and external medical records in standardized format using an external communicator module (900);
a. analyzing input data using a diagnostic module (400);
b. generating a treatment plan using a treatment module (700);
c. issuing electronic prescriptions or test requests;
d. adjusting treatment using a case monitoring module (800) based on monitored recovery data;
e. ordering prescriptions and scheduling lab tests automatically;
f. monitoring recovery through periodic data collection;
g. adjusting diagnosis and treatment protocols in real-time based on collected data;
h. initiating emergency services when critical health parameters are detected; and
i. integrating external lab results and images into the diagnostic module (400).
